(19) United States
(12) Patent Application Publication
(10) Pub. No.: US 2024/0191038 A1
(43) Pub. Date: Jun. 13, 2024

(54) FUNCTIONALIZED DIBLOCK COPOLYMER AND ITS PREPARATION METHOD AND APPLICATION

(71) Applicant: InnovatingBio (Shanghai) Co.,Ltd., Shanghai (CN)

(72) Inventors: Chun ZHOU, Shanghai (CN); Zhenxing YE, Shanghai (CN); Chenhong LU, Shanghai (CN)

(73) Assignee: InnovatingBio (Shanghai) Co.,Ltd., Shanghai (CN)

(21) Appl. No.: 18/028,220

(22) PCT Filed: Sep. 24, 2021
(86) PCT No.: PCT/CN2021/120152

§ 371 Date: Dec. 29, 2023

(30) Foreign Application Priority Data
Sep. 25, 2020 (CN) 202011023353.5

(51) Int. Cl. C08G 79/04; A61K 47/58; A61K 47/69; A61K 49/00

(52) U.S. Cl. CPC C08G 79/04; A61K 47/605; A61K 47/6935; A61K 49/0019; A61K 49/0054

(57) ABSTRACT

A functionalized diblock copolymer having the chemical structure shown in Formula I. The functionalized diblock copolymer or polymer particles can be widely used in tumor imaging, tumor therapy and other fields. It not only has good safety, realizes faster and adjustable degradation and removal of polymers (by changing the structure and number of functional groups) under acidic conditions, but also has excellent specific and high-quality imaging effects at the target site, with high signal-to-noise ratio, clear boundaries, long half-life, etc., which solves the problem of fluorescence imaging technology in real-time intraoperative navigation, and thus has a good industrialization prospect.

### FIELD OF THE INVENTION

The present disclosure relates to the field of organic chemistry, in particular, to a functionalized diblock copolymer and a preparation method and uses thereof. These uses mainly include uses for preparing tumor imaging probe reagents and uses for the manufacture of medicaments for the treatment of tumor.

### BACKGROUND OF THE INVENTION

Malignant tumors (cancers) have become one of the main reasons that threaten human lives and the threat has been increasing year by year. According to the 2019 National Cancer Report issued by the National Cancer Center of China, in China, malignant tumors have become one of the main public health problems that seriously threaten the health of the Chinese population. The latest statistics show that deaths from malignant tumors accounted for 23.91% of all deaths among residents. This resulted in medical expenses exceeding 220 billion RMB. In 2015, there were approximately 3.929 million cases of malignant tumors nationwide and 2.338 million cases of deaths.

Currently, standard treatments of cancer include surgical dissection, chemo-therapy, radiation therapy, and emerging immuno-therapy. Surgical removal of solid tumor is still the most effective and recommended first line of care treatment. Usually, surgeons rely on pre-operative imaging diagnosis, intra-operative clinical experience (including visual identification and palpation, etc.), and other clinical aids to determine the boundary of the tumor, for performing resection of the lesion during the surgical operation. However, because tumors are heterogeneously distributed tissues, and different types of tumors have different boundary characteristics, it is difficult to accurately determine tumor boundaries during surgery. Therefore, excessive surgical resection may seriously affect patients' post-operative quality of life (for example, total mastectomy for breast cancer; failure to preserve healthy parathyroid glands during thyroid cancer surgery; anal preservation problems caused by surgery for low rectal cancer, etc.). Insufficient resection is prone to recurrence (for example, non-invasive bladder cancer resection surgery has a high recurrence rate due to a high rate of tumor positive surgical margin). Consequently, accurately judging the boundaries of tumor lesions during surgery has become a key factor for the success of surgical operations and patient prognosis.

During the surgical procedure of tumor resection, the surgeon usually needs to decide whether to perform dissection of lymphatic tissue based on the pre-operative imaging diagnosis and the patient's pathological stage, and removes the cancerous tissue that may have metastasized into lymphatic tissue. In situations where pre-operative diagnosis is not definite, surgeons will choose to remove some of the patient's lymphatic tissues (adjacent lymph nodes), and during the operation (the patient is still under anesthesia), the pathology department will collect the specimens, perform a quick frozen pathological diagnosis and then provide the results back to the surgeon to decide on the necessity of further dissection of lymphatic tissue, and if yes the range and extent of dissection. Generally, the entire rapid frozen pathological examination process takes about 45 minutes to several hours. During this period, the medical team and medical resources in the operating room are all on standby, and the patient is also at increased risk of infection and prolonged anesthesia time while waiting in the operating room. Therefore, in addition to judging the boundary of the tumor, there is also a clinical need for a faster and more accurate pathological judgment of the tumor spreading tissue during the operation, shortening the operation time, accurately removing the cancer spreading tissue, reducing recurrence rate, and prolonging the patient survival after surgery.

In summary, the intra-operative imaging technology for solid tumors and metastatic tissues has great clinical significance. However, there are still great challenges for intra-operative specific imaging of cancer tissues. The main difficulties and corresponding current clinical development strategies are as follows:

1) The hardware should meet the requirements of the operating room.

Currently, widely used clinical imaging techniques such as X-ray scanning, CT (computed tomography), MRI (magnetic resonance imaging), ultrasound and PET-CT (positron emission computed tomography) are mainly used in preoperative tumor imaging diagnosis, but less for intraoperative tumor imaging diagnosis, due to the hardware requirements (such as volume), application requirements (such as electromagnetic fields) and many other reasons, which limit the real-time imaging diagnosis of these imaging technologies on the operating table and during the operation. In the prior art, because the intra-operative ultrasound imaging technology requires contact for imaging, its application in open tumor surgery is limited, and the imaging technology itself is based on tissue morphology with high false negatives and false positives. In brain tumor surgery, MRI scanning before surgery and constructing surgery coordinate information is also clinically applied during surgery, but this technology may affect the navigation quality of the surgery due to the deformation or displacement of the tissue from the time of image acquisition to the period of surgery.

Compared with the above-mentioned imaging technology, the technology based on fluorescence imaging has advantages in real-time application of surgery. First of all, the near-infrared (NIR) light source commonly used in fluorescent imaging technology has a stronger penetrating ability in tissues than visible light, ultraviolet light and other light sources, and is less affected by the main absorption chromophores inside the tissue, such as hemoglobin, oxygenated hemoglobin, and water. It can penetrate about 1 cm of tissue, and it has very important application value in tissue optical inspection, especially for shallow tissues. Secondly, the hardware implementation of fluorescence imaging can be more flexible. It can be designed as a movable imaging system integrating light sources of visible (white) light and NIR, imaging processing unit, and real-time imaging output monitor. Such imaging system can be configured for open surgery, or it can be designed as a small sterile probe with an external display screen to achieve white light and fluorescence endoscopic imaging system for minimally invasive surgery in the body. These two hardware designs have been approved by FDA and EMA (e.g., SPY Imaging system; PINPOINT® endoscopic fluorescence imaging system; da Vinci surgical robot system), and have been successfully applied in clinical surgery. Using a fluorescence microscope system, within 20 minutes after intra-operative intravenous injection of indocyanine green (ICG), ICG can be used to excite fluorescence under near-infrared light source for angiography (neurosurgery, vascular surgery, eye surgery, etc.). Methylene blue is also an approved fluorescent imaging agent and is used in some surgical procedures.

2) The intra-operative imaging technology should be specific to the tumor tissue.

The main requirements for achieving tumor specificity are: first of all, the targeted tumor type must have some specific characteristics. Some of the characteristics that are currently widely recognized are: specific surface receptors (such as folic acid, Her2/Neu, EGFR, PSMA and other receptors); characteristics of tumor microenvironment (specific metabolites, proteases; or acidic characteristics inside cancer cells (pHi: 5.0-6.0) or in the interstitial fluid between cells (pHe: 6.4-6.9), which are originated from the lactic acid metabolites produced by the aerobic glycolysis of the cancer cells after the rapid ingestion of glucose). Secondly, the above-mentioned specific characteristics should be used as a precise positioning target for the developed imaging technology, so as to effectively realize the specific accumulation of imaging agents at the tumor site. The usual means to achieve the accumulation at tumor sites are: using the specific receptors of cancer cells to achieve the specific binding of the imaging agent to them; using the acidity or other characteristics of the tumor microenvironment to retain and enrich the imaging agents at tumor site by chemical means; and using the enhanced permeability and retention effect (EPR) of tumor tissues to achieve selective local accumulation of some nanoparticles.

3) The imaging agent must be safe and can be degraded or eliminated from the body within a short period of time after use. There should be low tissue residue and no side effects. If a metabolic reaction occurs, the metabolites of the imaging agent should be harmless to human body.

The main clinical translation of intra-operative imaging technology for solid tumors uses the following types of technical means:

1) Folic Acid-Florescence Dye conjugate: On Target Laboratories has conducted a few clinical trials for conducting intra-operative tumor imaging guided surgeries for lung cancer and ovarian cancer. The advantage is that for these two selected tumor types, the target selection strategy is clear (except for individual tissues, folate receptors are expressed at very low levels on normal tissues, and overexpressed on the surface of some tumor cells). The disadvantage is that the application area is relatively narrow (only applicable to specific tumors with high folate receptor expression), and from the perspective of its imaging principles and clinical data, the quality of tumor specific imaging is somewhat limited (background contrast; the boundary between tumor and healthy tissue is not clearly distinguished), and the reason may be that imaging molecules that normally circulate in the body (not bound to tumor receptors) can fluoresce when illuminated by the excitation light source, causing background fluorescence, or false positive images of non-tumor sites (“off-target” phenomenon, for example, in some healthy tissues such as kidneys, there are also different degrees of folate receptors), or may be that the expression of folic acid in tumor tissues may not be completely uniform due to the heterogeneity of tumors mentioned above, causing defects in image quality. From the clinical data, the clearance of the background is related to the dosage. Basically, it takes 24 hours to 4 days for complete clearance. The effect of tumor imaging (tumor/normal tissue ratio, abbreviated as TNR, is 2-3) is acceptable but improvement could be desired.

2) Antibody (mAB)—Florescence Dye conjugate: There have been several clinical trials for intro-operative imaging navigation of tumors such as glioma (mAB targeting EGFR receptors, Cetuximab) and colon cancer, lung cancer (targeting CEA receptor). Compared with folic acid-florescence imaging molecule conjugate design, the antibody molecule used for targeting has good biocompatibility, and the circulation cycle in the body is very long (3-7 days). For the selected tumor type, the target is clear and the binding mechanism is clear. Its shortcomings are also obvious. The long circulation time of antibody molecules will also cause high background fluorescence. It also has other problems, such as narrow application range (only applicable to tumors with high expression of specific receptors). For example, there are false positive images of non-tumor sites (the selected target may exist in healthy tissues), and the non-uniform characteristics of the tumor mentioned above. From the clinical and animal research data, the tumor imaging effect of this technology (tumor/normal tissue ratio, abbreviated as TNR, is 2-5) is acceptable, but the image is usually accompanied by strong background fluorescence.

3) Peptide-Florescence Dye conjugate: In view of the characteristics of several tumor cells and tumor microenvironment mentioned above, polypeptides can be used for selective targeting to target fluorescent imaging molecules to tumor sites. At present, there are several designs in the direction of research and development and clinical transformation: R. Tsien and Avelas Biosciences, Inc. use a special U-shaped polypeptide combination design. One end of the polypeptide is positively charged under physiological conditions (this end of the polypeptide is linked to a fluorescent imaging molecule), the other end of the polypeptide is negatively charged under physiological conditions, and the two ends of the polypeptide are connected by a linker, which can be cleaved by the protease present in the tumor microenvironment. After the disconnection, the polypeptide with the fluorescent imaging molecule exhibits a positive charge, which can be attracted to the negative charge on the surface of the cancer cell and then adsorbed on the surface. Later, it enters the cancer cell through the endocytosis mechanism, and then the imaging agent molecule that enters the cancer cell can emit fluorescence under the illumination of the excitation light source. It can be seen that after this type of imaging agent enters the body, the time window for completing this series of action within a limited time (even with PEG modification, the circulation half-life is only around 20 minutes) is not sufficient, resulting in poor imaging results (the TNR is 2-3). The team of Donald M. Engelman of Yale University proposed a different design to form a conjugate between a fluorescent molecule and a polypeptide. The signal targeted by the polypeptide is the acidic characteristic of the tumor microenvironment. Under normal physiological conditions, the polypeptide is negatively charged, but it becomes neutral in an acidic environment. Under electrically neutral conditions, the lipophilicity of the polypeptide increases, which drives the deposition and transmembrane behavior of the polypeptide on the surface of cancer cells to achieve the specific enrichment of fluorescent molecules at the tumor site. From the results of live imaging, this technology has achieved good tumor imaging quality (TNR is about 6), but the error range of the reported data is too large and the effect is not good. Lumicell's design is to connect a fluorescent imaging molecule and another molecule that can absorb fluorescence through a peptide. The selected peptide can be cleaved under the catalysis of some common proteases (such as Cathepsin K, L, S) in the tumor microenvironment, so that the fluorescent molecules and the molecules that actively absorb fluorescence will be separated and then fluoresce in the presence of the excitation light source. This design can reduce the background fluorescence during the cycle, because the entire imaging agent molecule does not fluoresce before it reaches the tumor microenvironment. By conjugating a strand of polyethylene glycol (PEG), the blood circulation time can be achieved to about 24 hours, and the tumor image quality (TNR is 3-5) is only acceptable. In addition, another disadvantage of this technology is whether the selected peptide sequence can achieve high specific tumor targeting.

4) Dye-carrying nano-particles (NP): In the field of medical imaging, nanoparticles are widely used. The main categories are liposomal nanoparticles, inorganic nanoparticles, and polymer nanoparticles. DEFINITY® is a phospholipid liposome of Lantheus Medical (now BMS) approved in 2001, and is used to stabilize perfluoropropane (C3F8) bubbles and used as an ultrasound imaging agent. There are many types of inorganic nanoparticles (silica; iron oxide; quantum dots; carbon nanotubes, etc.). Generally, the clinical application difficulty of inorganic nanoparticles is safety. However, it is often difficult to achieve specific tumor fluorescence imaging if fluorescent groups are introduced merely by chemical modification on the surface of nanoparticles. U. Wiesner and others have successfully advanced several early clinical studies. The use of small particle size (5-20 nm) SiO₂ nanoparticles allows the used nanoparticles to be removed from the kidney to improve safety, and the core of the nanoparticles is embedded with fluorescent molecules, and the introduction of specific targeting groups on the surface of nanoparticles can achieve specific tumor fluorescence imaging. The fluorescent molecules introduced by this method can overcome the possible defects of fluorescence quenching of conventional nanoparticle-fluorescent molecule conjugates that may occur due to long residence time in the body, but the reported half-life is short (10-30 minutes). The tumor imaging effect is acceptable, and its TNR is 5-10 (the reported data has a large error range), but the liver absorption is also very high (the tumor/liver ratio is about 2). The author believes that although small-sized nanoparticles (less than 20 nm) can be eliminated by the kidneys, they still do not rule out their clinical risks (such as spreading to the brain through the BBB, etc.). The typical structure of polymer nanoparticles is to use amphiphilic diblock polymers, such as PEG-PLGA, PEG-PEG-Glutamate, and PEG-Aspartate, which are several types of clearable (PEG)/degradable (another block) polymers that are currently working to the clinic. Building on the previous work of Langer et al. on pH-responsive polymer microspheres (the polymer backbone contains amino groups that can be protonated at pH 6.5), Kim et al. introduced PEG blocks to construct pH-responsive amphiphilic diblock copolymer. The diblock copolymer realizes the dissolution of nanoparticles in the weakly acidic environment of the tumor (the nanoparticle core is ionized in the acidic environment, and the charge repulsive force is generated, which destroys the energy balance of the amphiphilic self-assembly).

### SUMMARY

The present disclosure provides a functionalized diblock copolymer and a preparation method and uses thereof.

An aspect of the present disclosure provides a functionalized diblock copolymer. The chemical structure of the functionalized diblock copolymer is shown in Formula I:

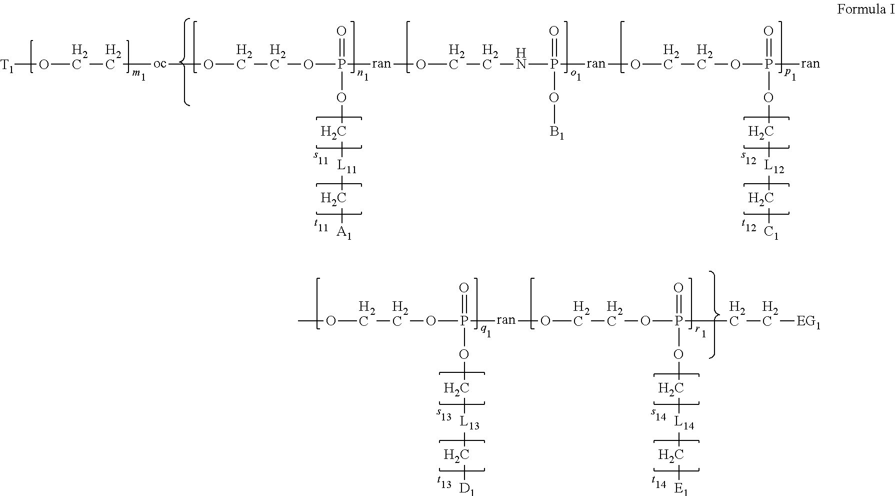

In formula I, m₁=22˜1136, n₁=10˜500, o₁=0˜50, p₁=0.5˜50, q₁=0˜500 and r₁=0˜200;
s₁₁=1˜10, s₁₂=1˜10, s₁₃=1˜10, s₁₄=1˜10;
t₁₁=1˜10, t₁₂=1˜10, t₁₃=1˜10, t₁₄=1˜10;
L₁₁, L₁₂, L₁₃ and L₁₄ are linking groups;
A₁ is selected from protonatable groups;
B₁ is selected from degradability-regulating groups;
C₁ is selected from fluorescent molecular groups;
D₁ is selected from delivery molecular groups;
E₁ is selected from hydrophilic/hydrophobic groups;
T₁ is selected from capping groups;
EG₁ is selected from capping groups.

In formula I, -co- denotes block copolymers, -ran- denotes random distribution of units with different side chains within the polymer block separated by -co-.

Another aspect of the present disclosure provides polymer particles prepared from the above-mentioned functionalized diblock copolymer.

Another aspect of the present disclosure provides the use of the aforementioned functionalized diblock copolymer or the aforementioned polymer particles in the preparation of imaging probe reagents and pharmaceutical preparations.

Another aspect of the present disclosure provides a composition including the aforementioned functionalized diblock copolymer or the aforementioned polymer particles.

### BRIEF DESCRIPTION OF THE DRAWINGS

FIG. 1 shows a schematic diagram of the pKa measurement results of different tertiary amine attached to the side chain of a polymer (mPEG-PPE90) in example 2 of the present disclosure.

FIG. 2 shows a schematic diagram of the critical micelle concentration (CMC) measurement results of PPE90-TPrB (left) and PPE200-TPrB (right) in example 3 of the present disclosure.

FIGS. 3a-3d show schematic diagrams of dynamic light scattering (DLS) and transmission electron microscopy (TEM) test results of PPE90-TPrB-ICG nanoparticles in PBS buffer solution in example 4 of the present disclosure, where FIG. 3a and FIG. 3c are the DLS and TEM test results of PBS (pH 8.0) respectively, FIG. 3b and FIG. 3d are the DLS and TEM test results of PBS (pH 6.0), respectively.

FIGS. 4a-4h show schematic diagrams of the fluorescence test results in example 5 of the present disclosure, where FIG. 4a is the summary of the relationship between the fluorescence emission intensity of PPE-TPrB-ICG3 with different degrees of polymerization (DP) at 821 nm and pH, FIGS. 4b-4h are fluorescence emission spectra of PPE-TPrB-ICG3 with different DP in various PBS buffers, FIG. 4b: DP 70, FIG. 4c: DP 90, FIG. 4d: DP 120, FIG. 4e: DP 150, FIG. 4f: DP 200, FIG. 4g: DP 250, FIG. 4h: DP 300.

FIGS. 5a-5d show schematic diagrams of the fluorescence test results in example 5 of the present disclosure, in which FIGS. 5a-5d are fluorescence emission spectra of PPE-TPrB-ICG3, PPE-TPrB-C520-ICG3, PPE-TPrB-C940-ICG3, PPE-TPrB-C980-ICG3, respectively, in various PBS buffers.

FIGS. 6a-6e show schematic diagrams of the fluorescence test results in Example 5 of the present disclosure, in which FIG. 6a shows the relationship between the normalized fluorescence intensity of PPE90-ICG3 with different tertiary amine side chains at 821 nm and the solution pH, FIGS. 6b-6e are the fluorescence emission spectra of fluorescent probes in buffer solutions with different pH, FIG. 6b: TPrB; FIG. 6c: TBB; FIG. 6d: TPePe; FIG. 6e: THH.

### DETAILED DESCRIPTION

In order to make the purpose of the present disclosure, technical solutions and beneficial technical effects of the present disclosure clearer, the invention will be further described in detail below in conjunction with examples. Those skilled in the art can easily understand other advantages and effects of this invention from the content disclosed in this specification.

In the present disclosure, “diblock copolymer” generally refers to a polymer having two different polymer segments (as if two blocks linked together) with different chemical compositions.

In the present disclosure, the “protonatable group” generally refers to a group that can combine with a proton, that is, it can bind at least one proton. These groups usually have a lone pair of electrons, so that at least one proton can be combined with the protonatable group.

In the present disclosure, “degradability regulating group” is a type of group that can change the degradability of a compound in vivo.

In the present disclosure, “fluorescent molecular group” generally refers to a type of group corresponding to fluorescent molecules. Compounds containing these groups can usually have characteristic fluorescence in the ultraviolet-visible-near infrared regions, and their fluorescent properties (excitation and emission wavelengths, intensity, lifetime, polarization, etc.) can change with the nature of the environment.

In the present disclosure, “delivery molecular group” usually means various molecules that can be chemically bonded to the main chain of the block copolymer through a side chain, or interact with the hydrophobic side chain groups of the block copolymer through physical force (such as charge forces, hydrogen bonding, van der Waals force, hydrophobic interaction, etc.) and can be delivered by nanoparticles formed by self-assembly of the block polymer in aqueous solution.

In the present disclosure, “hydrophilic/hydrophobic group” generally refers to a group with a certain degree of hydrophilicity or lipophilicity.

In the present disclosure, “alkyl” usually refers to a saturated aliphatic group, which can be linear or branched. For example, C1-C20 alkyl usually refers to alkyl groups with 1, 2, 3, 4, 5, 6, 7, 8, 9, 10, 11, 12, 13, 14, 15, 16, 17, 18, 19, or 20 carbon atom(s). Specific alkyl groups can include, but are not limited to, methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, eicosyl.

In the present disclosure, “alkenyl” generally refers to an unsaturated aliphatic group with C═C bond(s) (carbon-carbon double bonds, ethylenic bonds), which can be straight or branched. For example, C2-C10 alkenyl generally refers to alkenyl groups of 2, 3, 4, 5, 6, 7, 8, 9, or 10 carbon atoms. Specific alkenyl groups may include, but are not limited to, vinyl, propenyl, butenyl, pentenyl, hexenyl, heptenyl, octenyl, nonenyl, and decenyl.

In the present disclosure, “alkynyl” generally refers to an unsaturated aliphatic group with C≡C bond (s) (carbon-carbon triple bonds, acetylene bonds), which can be straight or branched. For example, C2-C10 alkynyl generally refers to alkynyl groups of 2, 3, 4, 5, 6, 7, 8, 9, or 10 carbon atoms. Specific alkynyl groups may include, but are not limited to, ethynyl, propynyl, butynyl, pentynyl, hexynyl, heptynyl, octynyl, nonynyl, and decynyl.

In the present disclosure, “cycloalkyl” generally refers to saturated and unsaturated (but not aromatic) cyclic hydrocarbons. For example, C3-C10 cycloalkyl generally refers to cycloalkyl groups of 3, 4, 5, 6, 7, 8, 9, or 10 carbon atoms. Specific cycloalkyl groups may include, but are not limited to, cyclopropyl, cyclobutyl, cyclopentyl, cyclohexyl, cycloheptyl, cyclooctyl, cyclononyl, cyclodecyl. The term “cycloalkyl” in the present disclosure also includes saturated cycloalkyls in which optionally at least one carbon atom can be replaced by a heteroatom, which can be selected from S, N, P, and O. In addition, a monounsaturated or polyunsaturated (preferably monounsaturated) cycloalkyl group without heteroatoms in the ring should belong to the term cycloalkyl group as long as it is not an aromatic system.

In the present disclosure, “aromatic group” generally refers to a ring system with at least one aromatic ring and no heteroatoms. The aromatic group may be substituted or unsubstituted. The specific substituent may be selected from C1-C6 alkyl, C1-C6 alkoxy, C3-C10 cycloalkyl, hydroxyl, halogen, etc. Specific aromatic groups may include, but are not limited to, phenyl, phenol, aniline, and the like.

In the present disclosure, “heteroaryl” generally refers to a ring system having at least one aromatic ring and optionally one or more (for example, 1, 2, or 3) heteroatoms selected from nitrogen, oxygen, and sulfur. The heteroaryl group may be substituted or unsubstituted, and the specific substituent may be selected from C1-C6 alkyl, C1-C6 alkoxy, C3-C10 cycloalkyl, hydroxyl, halogen and the like. Specific heteroaryl groups may include, but are not limited to, furan, benzofuran, thiophene, benzothiophene, pyrrole, pyridine, pyrimidine, pyridazine, pyrazine, quinoline, isoquinoline, phthalazine, benzo-1,2,5-thiadiazole, benzothiazole, indole, benzotriazole, benzodioxolane, benzodioxane, benzimidazole, carbazole, or quinazoline.

In the present disclosure, “targeting agents” generally refer to agents that can specifically direct a specific compound to a desired site of action (target area), which may be in the form of polymeric particles that typically have relatively low, no, or almost no interaction with non-target tissues.

In the present disclosure, “imaging probe” generally refers to a class of substances that can enhance the effect of image observation after being injected (or taken) into human tissues or organs.

In the present disclosure, “individual” generally includes humans and non-human animals, such as mammals, dogs, cats, horses, sheep, pigs, cows, and the like.

After a lot of practical research, the inventors of the present disclosure has provided a class of functionalized diblock copolymers. These diblock copolymers can be pH-responsive and degradable under corresponding pH conditions through innovative chemical modification strategies. Therefore, it can be used as in various fields utilizing such said features, and the present disclosure has been completed on this basis.

The first aspect of the present disclosure provides a functionalized diblock copolymer having the chemical structural formula shown below:

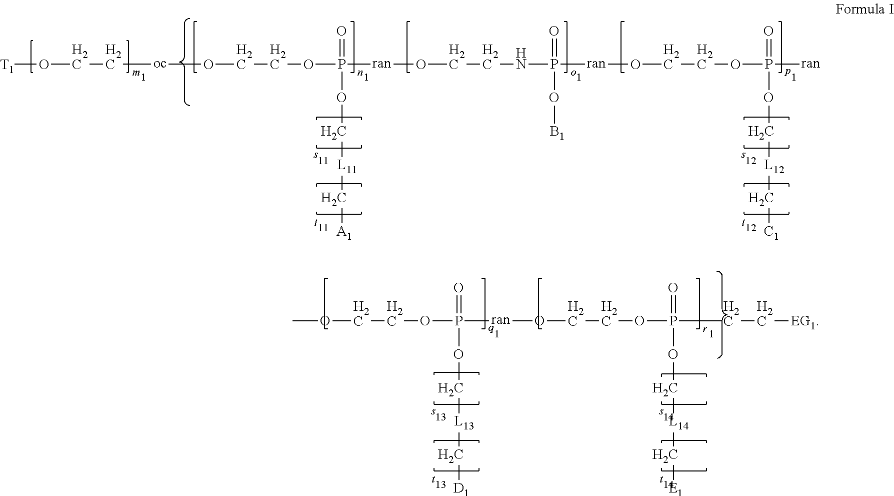

In formula I, m₁=22˜1136, n₁=30˜500, o₁=0˜50, p₁=0.5˜50, q₁=0˜500 and r₁=0˜200;
s₁₁=1˜10, s₁₂=1˜10, s₁₃=1˜10, s₁₄=1˜10;
t₁₁=1˜10, t₁₂=1˜10, t₁₃=1˜10, t₁₄=1˜10;
L₁₁, L₁₂, L₁₃ and L₁₄ are linking groups;
A₁ is selected from protonatable groups;
B₁ is selected from degradability-regulating groups;
C₁ is selected from fluorescent molecular groups;
D₁ is selected from delivery molecular groups;
E₁ is selected from hydrophilic/hydrophobic groups;
T₁ is selected from capping groups;
EG₁ is selected from capping groups.

The compound of formula I is a diblock copolymer of polyethylene glycol-polylactone, wherein the side chain structure of the polylactone block is randomly distributed, and the general formula is represented by ran.

In the compound of formula I, L₁₁, L₁₂, L₁₃, L₁₄ are usually linking groups, which is mainly used to link the main chain of the functionalized diblock copolymer and its pendant side chains. In a specific example of the present disclosure, L₁₁, L₁₂, L₁₃, L₁₄ can be independently selected from —S—, —O—, —OC(O)—, —C(O)O—, —SC(O)—, —C(O)—, —OC(S)—, —C(S)O—, —SS—, —C(R₁)═N—, —N═C(R₂)—, —C(R₃)═N—O—, —O—N═C(R₄)—, —N(R₅)C(O)—, —C(O)N(R₆)—, —N(R₇)C(S)—, —C(S)N(R₈)—, —N(R₉)C(O)N(R₁₀)—, —OS(O)O—, —OP(O)O—, —OP(O)N—, —NP(O)O—, —NP(O)N—, wherein, R₁˜R₁₀ are each independently selected from H, C1-C10 alkyl, and C3-C10 cycloalkyl.

In another specific embodiment of the present disclosure, L₁₁, L₁₂, L₁₃, and L₁₄ may be independently S.

In the compound of formula I, A₁ is usually selected from protonatable groups, and this group and the block of the polymer in which this group is located are mainly used to adjust the pH response of the polymer. In an embodiment of the present disclosure, A₁ can be

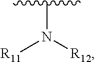

wherein, R₁₁ and R₁₂ are each independently selected from C1-C10 alkyl, C2-C10 alkenyl, C2-C10 alkynyl, C3-C10 cycloalkyl, and aryl. In another embodiment of the present disclosure, A₁ can be

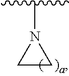

wherein, a=1-10, and a is a positive integer.

In another embodiment of the present disclosure, A₁ can be

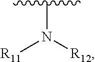

wherein R₁₁ is n-propyl, R₁₂ is n-butyl. In another embodiment of the present disclosure, A₁ can be

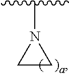

wherein, a=1-10, and a is a positive integer.

In the compound of formula I, B₁ is usually a degradability-regulating group, and this group and the block of the polymer in which this group is located are mainly used to regulate the in vivo degradability of the polymer. In a specific embodiment of the present disclosure, B₁ can be selected from C1-C18 alkyl groups, cations and the like, and the cations can be Li⁺, Na⁺, K⁺, Ca²⁺, Zn²⁺, Fe²⁺, Fe³⁺, Mg²⁺, Zn²⁺, NH⁴⁺ and the like.

In another embodiment of the present disclosure, B₁ may be methyl.

In the compound of formula I, C₁ is usually a fluorescent molecular group, and this group and the block of the polymer in which this group is located are mainly used to introduce fluorescent molecular groups. The fluorescent molecular group may specifically include, but is not limited to, one or a combination of organic reagents, metal chelate and the like. In an embodiment of the present disclosure, C₁ may include fluorescent molecules such as ICG (Indocyanine Green), METHYLENE BLUE, CY3.5, CY5, CY5.5, CY7, CY7.5, BDY630, BDY650, BDY-TMR, Tracy 645, and Tracy 652.

In another embodiment of the present disclosure, C₁ may include indocyanine green (ICG), and ICG may be connected to the side chain of the block through an amide bond.

In the compound of formula I, D₁ can be a delivery molecular group, and this group and the block of the polymer in which this group is located are mainly used to introduce various molecular groups that can be delivered through a block copolymer. These molecular groups may include, but are not limited to, fluorescence quenching groups, drug molecule groups (for example, photodynamic therapy precursor molecules, chemotherapeutic drug molecules, biopharmaceutical molecules, etc.) and the like. In an embodiment of the present disclosure, the fluorescence quenching group can be selected from BHQ-0, BHQ-1, BHQ-2, BHQ-3, BHQ-10, QXL-670, QXL-610, QXL-570, QXL 520, QXL-490, QSY35, QSY7, QSY21, QXL 680, Iowa Black RQ, Iowa Black FQ. In an embodiment of the present disclosure, the drug molecule group can be a group corresponding to chemotherapeutic drugs, which can specifically be nucleic acid drugs, paclitaxel, cisplatin, doxorubicin, irinotecan, SN38 and other groups corresponding to drug molecules. In another embodiment of the present disclosure, the drug molecule group can be a group corresponding to photodynamic therapy chemical drugs, and may specifically be a group corresponding to 5-ALA and its derivative structure (lipidation or fatty chaining, etc.). The specific chemical structure of the group is as follows:

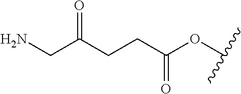 [SMILES: *OC(=O)CCC(=O)CN]

In the compound of formula I, E₁ can be a hydrophilic/hydrophobic group, and this group and the block of the polymer in which this group is located are mainly used to adjust the hydrophobicity/hydrophilicity of the hydrophobic block of the polymer. In an embodiment of the present disclosure, E₁ can be selected from H, C1-C18 alkyl, —O—R₁₁, —S—R₁₂, wherein R₁₁˜R₁₂ are each independently selected from H, C1-C18 alkyl, C3-C10 cycloalkyl, and aryl.

In an embodiment of the present disclosure, E₁ can be selected from n-pentyl and n-nonyl.

In the compound of formula I, T₁ can usually be selected from end groups of polyethylene glycol (PEG) initiators. In an embodiment of the present disclosure, T₁ can be selected from —CH₃, and —H.

In the compound of formula I, EG₁ can usually be produced by different capping agents added after polymerization. In a specific embodiment of the present disclosure, EG₁ can be —Y—R₁₃, wherein Y is selected from O, S, and N, and R₁₃ is selected from H, C1-C20 alkyl, C3-C10 cycloalkyl, and aryl.

In another embodiment of the present disclosure, EG₁ can be —OH.

In the compound of formula I, the molecular weight of polyethylene glycol (PEG) block can be in a range of 1000˜50000 Da, 1000˜2000 Da, 2000˜3000 Da, 3000˜4000 Da, 4000˜5000 Da, 5000˜6000 Da, 6000˜7000 Da, 7000˜8000 Da, 8000˜9000 Da, 9000˜10000 Da, 10000˜12000 Da, 12000˜14000 Da, 14000˜16000 Da, 16000˜18000 Da, 18000˜20000 Da, 22000˜24000 Da, 24000˜26000 Da [symbol] 26000˜28000 Da [symbol] 28000˜30000 Da, 30000˜32000 Da, 32000˜34000 Da, 34000˜36000 Da, 36000˜38000 Da [symbol] 38000˜40000 Da, 40000˜42000 Da, 42000˜44000 Da, 44000˜46000 Da, 46000˜48000 Da, or 48000˜50000 Da; the molecular weight of polyphosphate (PPE) block can generally be in a range of 5000˜50000 Da [symbol] 5000˜6000 Da [symbol] 6000˜7000 Da, 7000˜8000 Da, 8000˜9000 Da, 9000˜10000 Da, 10000˜12000 Da [symbol] 12000˜14000 Da, 14000˜16000 Da, 16000˜18000 Da, 18000˜20000 Da, 22000˜24000 Da, 24000˜26000 Da, 26000˜28000 Da, 28000˜30000 Da, 30000˜32000 Da, 32000˜34000 Da, 34000˜36000 Da, 36000˜38000 Da, 38000˜40000 Da, 40000˜42000 Da, 42000˜44000 Da, 44000˜46000 Da, 46000˜48000 Da, or 48000˜50000 Da. In the present disclosure, the molecular weight of a block usually refers to the molecular weight of the main chain molecule in this block, and the molecular weight is usually the number-average molecular weight (Mn).

In a specific embodiment, the molecular weight of the polyethylene glycol block can be in a range of 2000˜10000 Da, and the molecular weight of the polyphosphate block can be in a range of 6000˜37000 Da.

In the compound of formula I, m₁ can be in a range of 22˜1136, 22˜32, 32˜42, 42˜52, 52˜62, 62˜72, 72˜82, 82˜92, 92˜102, 102˜122, 122˜142, 142˜162, 162˜182, 182˜202, 202˜242, 242˜282, 282˜322, 322˜362, 362˜402, 402˜442, 442˜482, 482˜522, 522˜562, 562˜602, 602˜642, 642˜682, 682˜722, 722˜762, 762˜802, 802˜842, 842˜882, 882˜902, 902˜942, 942˜982, or 982˜1136.

n₁ can be in a range of 10˜500, 10˜15, 15˜20, 20˜25, 25˜30, 30˜35, 35˜40, 40˜45, 45˜50, 45˜50, 50˜60, 60˜70, 70˜80, 80˜90, 90˜100, 100˜120, 120˜140, 140˜160, 160˜180, 180˜200, 200˜220, 220˜240, 240˜260, 260˜280, 280˜300, 300˜320, 320˜340, 340˜360, 360˜380, 380˜400, 400˜420, 420˜440, 440˜460, 460˜480, or 480˜500.

o₁ can be in a range of 0˜50, 0˜1, 1˜2, 2˜4, 4˜6, 6˜8, 8˜10, 10˜12, 12˜14, 14˜16, 16˜18, 18˜20, 20˜25, 25˜30, 30˜35, 35˜40, 40˜45, or 45˜50.

p1 can be in a range of 0.5˜50, 0.5-1, 1˜2, 2˜3, 3˜4, 4˜5, 6˜7, 6˜7, 7˜8, 8˜9, 9˜10, 10˜12, 12˜14, 14˜16, 16˜18, 18˜20, 20˜25, 25˜30, 30˜35, 35˜40, 40˜45, or 45˜50.

q₁ can be in a range of 0˜500, 0˜1, 1˜2, 2˜4, 4˜6, 6˜8, 8˜10, 10˜12, 12˜14, 14˜16, 16˜18, 18˜20, 20˜25, 25˜30, 30˜35, 35˜40, 40˜45, 45˜50, 45˜50, 50˜60, 60˜70, 70˜80, 80˜90, 90˜100, 100˜120, 120˜140, 140˜160, 160˜180, 180˜200, 200˜220, 220˜240, 240˜260, 260˜280, 280˜300, 300˜320, 320˜340, 340˜360, 360˜380, 380˜400, 400˜420, 420˜440, 440˜460, 460˜480, or 480˜500.

r₁ can be in a range of 0˜200, 0˜1, 1˜2, 2˜4, 4˜6, 6˜8, 8˜10, 10˜12, 12˜14, 14˜16, 16˜18, 18˜20, 20˜25, 25˜30, 30˜35, 35˜40, 40˜45, 45˜50, 45˜50, 50˜60, 60˜70, 70˜80, 80˜90, 90˜100, 100˜120, 120˜140, 140˜160, 160˜180, or 180˜200.

s₁₁ can be in a range of 1˜10, 1˜2, 2˜3, 3˜4, 4˜5, 6˜7, 6˜7, 7˜8, 8˜9, or 9˜10.

s₁₂ can be in a range of 1˜10, 1˜2, 2˜3, 3˜4, 4˜5, 6˜7, 6˜7, 7˜8, 8˜9, or 9˜10.

s₁₃ can be in a range of 1˜10, 1˜2, 2˜3, 3˜4, 4˜5, 6˜7, 6˜7, 7˜8, 8˜9, or 9˜10.

s₁₄ can be in a range of 1˜10, 1˜2, 2˜3, 3˜4, 4˜5, 6˜7, 6˜7, 7˜8, 8˜9, or 9˜10.

t₁₁ can be in a range of 1˜10, 1˜2, 2˜3, 3˜4, 4˜5, 6˜7, 6˜7, 7˜8, 8˜9, or 9˜10.

t₁₂ can be in a range of 1˜10, 1˜2, 2˜3, 3˜4, 4˜5, 6˜7, 6˜7, 7˜8, 8˜9, or 9˜10.

t₁₃ can be in a range of 1˜10, 1˜2, 2˜3, 3˜4, 4˜5, 6˜7, 6˜7, 7˜8, 8˜9, or 9˜10.

t₁₄ can be in a range of 1˜10, 1˜2, 2˜3, 3˜4, 4˜5, 6˜7, 6˜7, 7˜8, 8˜9, or 9˜10.

In a specific embodiment, in Formula I, m₁=22-1136, n₁=10-500, o₁=0, p₁=0.5-50, q₁=0 and r₁=0. For products prepared by these polymers (for example, polymer particles), the fluorescent molecules distributed in the hydrophobic core do not emit light under certain excitation conditions (e.g., with NIR as the excitation light source) due to the FRET (Fluorescence Resonance Energy Transfer) effect. However, after being administered to an individual, the polymer particles can be enriched at the target site (for example, tumor site) through EPR (Enhanced Permeation and Retention) passive targeting (or other tissue uptake methods). Because the target site has a special pH environment (for example, acidic environment), the protonatable group (i.e., the A₁ group) can be protonated in this pH range, and the charge repulsion generated by its protonation and the increase in polymer solubility drive the disintegration of the polymer particles. The FRET effect of the fluorescent group on the molecular segment is reduced or even completely eliminated. The polymer molecules in the dispersed state enriched in the target site can emit fluorescence under certain excitation conditions (for example, with the near-infrared ray as the excitation light source).

In a preferred embodiment, the chemical structural formula of the functionalized diblock copolymer is shown as one of the following:

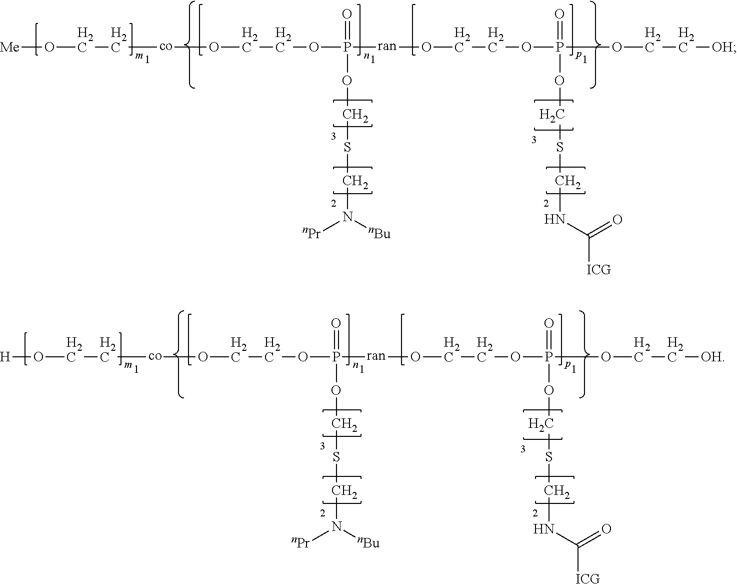

In another preferred embodiment of the present disclosure, m₁=44-226, n₁=50-300, and p₁=1-5.

In a specific embodiment of the present disclosure, in Formula I, m₁=22˜1136, n₁=10˜500, o₁=0, p1=0.5˜50, q₁=0, and r₁=1˜200. For the products prepared by these polymers (for example, polymer particles), the fluorescent molecules distributed in the hydrophobic core do not emit light under certain excitation conditions (for example, in the case of near infrared ray as the excitation light source) due to the FRET effect. The addition of hydrophilic/hydrophobic groups (i.e., E1 groups) increases the stability of polymer particles, enhances the FRET effect of polymer particles (more complete fluorescence quenching), and changes the acidity sensitivity of polymer particles. However, after being administered to an individual, the polymer particles can be enriched at the target site (for example, tumor site) through EPR passive targeting (or other tissue uptake methods). Because the target site has a special pH environment (for example, acidic environment), the protonatable group (i.e., the A₁ group) can be protonated in this pH range, and the charge repulsion generated by its protonation and the increase in polymer solubility drive the disintegration of the polymer particles. The FRET effect of the fluorescent group on the molecular segment is reduced or even completely eliminated. The polymer molecules in the dispersed state enriched in the target site can emit fluorescence under certain excitation conditions (for example, with the near-infrared ray as the excitation light source).

In a preferred embodiment, the chemical structural formula of the functionalized diblock copolymer is shown as one of the following:

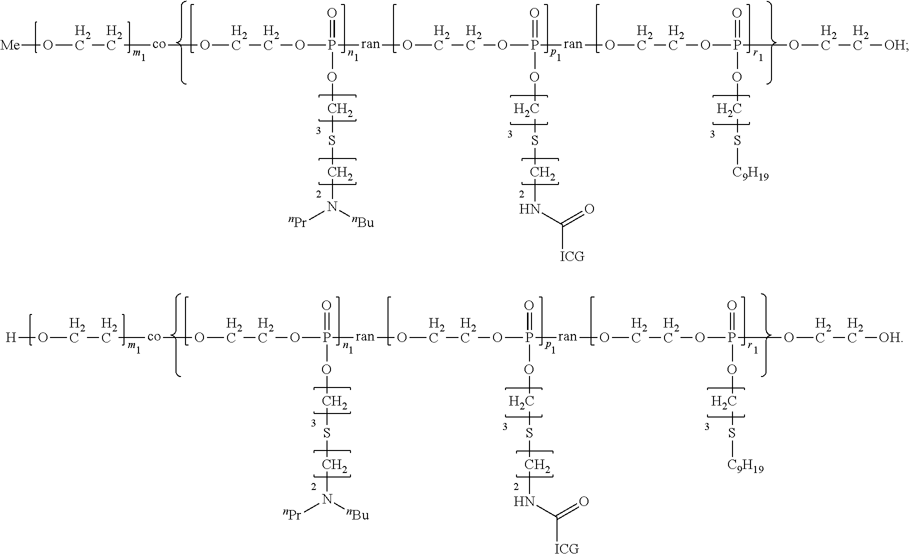

In another preferred embodiment, m₁=44˜226, n₁=70˜300, p₁=0.5˜5, and r₁=10˜100.

In a specific embodiment, in Formula I, m₁=22˜1136, n₁=10˜500, o₁=1˜50, p₁=0.5˜50, q₁=0, and r₁=0. For the products prepared by these polymers (for example, polymer particles), the fluorescent molecules distributed in the hydrophobic core do not emit light under certain excitation conditions (for example, in the case of near infrared ray as the excitation light source) due to the FRET effect. The addition of the degradability-regulating group (i.e., B₁ group) can adjust the degradation performance of the polymer in vivo. However, after being administered to an individual, the polymer particles can be enriched at the target site (for example, tumor site) through EPR passive targeting (or other tissue uptake methods). Because the target site has a special pH environment (for example, acidic environment), the protonatable group (i.e., the A1 group) can be protonated in this pH range, and the charge repulsion generated by its protonation and the increase in polymer solubility drive the disintegration of the polymer particles. The FRET effect of the fluorescent group on the molecular segment is reduced or even completely eliminated. The polymer molecules in the dispersed state enriched in the target site can emit fluorescence under certain excitation conditions (for example, with the near-infrared ray as the excitation light source).

In a preferred embodiment, the chemical structural formula of the functionalized diblock copolymer is shown as one of the following:

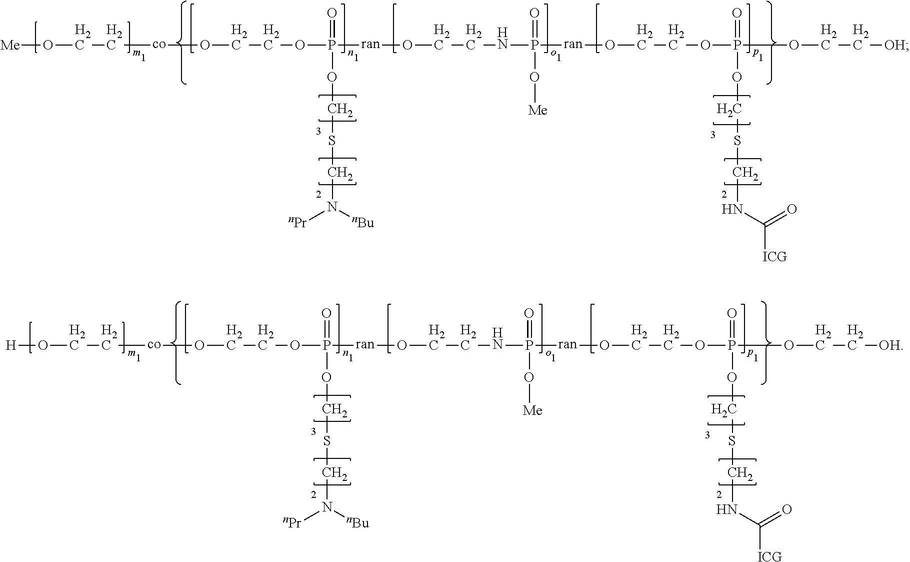

In another preferred embodiment, m₁=44˜226, n₁=70˜300, o₁=1˜10, and p₁=0.5˜5.

In a specific embodiment, in Formula I, m₁=22˜1136, n₁=10˜500, o₁=1˜50, p₁=0.5˜50, q₁=0, and r₁=1˜200. For the products prepared by these polymers (for example, polymer particles), the fluorescent molecules distributed in the hydrophobic core do not emit light under certain excitation conditions (for example, in the case of near infrared ray as the excitation light source) due to the FRET effect. The addition of hydrophilic/hydrophobic groups (i.e., E₁ groups) increases the stability of polymer particles, enhances the FRET effect of polymer particles (more complete fluorescence quenching), and changes the acidity sensitivity of polymer particles. The introduction of the degradability-regulating group (i.e., B1 group) can adjust the degradation performance of the polymer in vivo. However, after being administered to an individual, the polymer particles can be enriched at the target site (for example, tumor site) through EPR passive targeting (or other tissue uptake methods). Because the target site has a special pH environment (for example, acidic environment), the protonatable group (i.e., the A1 group) can be protonated in this pH range, and the charge repulsion generated by its protonation and the increase in polymer solubility drive the disintegration of the polymer particles. The FRET effect of the fluorescent group on the molecular segment is reduced or even completely eliminated. The polymer molecules in the dispersed state enriched in the target site can emit fluorescence under certain excitation conditions (for example, with the near-infrared ray as the excitation light source).

In a preferred embodiment, the chemical structural formula of the functionalized diblock copolymer is shown as one of the following:

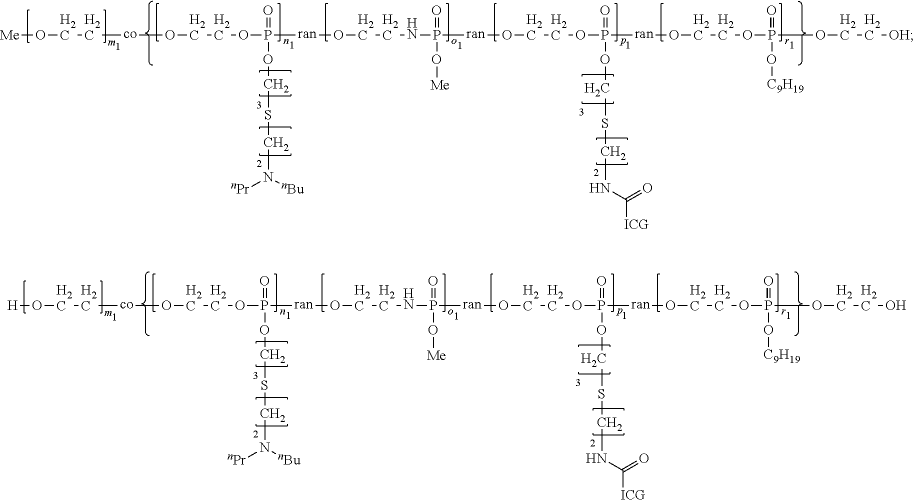

In another preferred embodiment, m₁=44˜226, n₁=50˜300, o₁=1˜10, p₁=0.5˜5, and r₁=10˜100.

In a specific embodiment, in Formula I, m₁=22˜1136, n₁=10˜500, o₁=1˜50, p₁=0.5˜50, g₁=1˜500, and r₁=0. For the products prepared by these polymers (for example, polymer particles), the fluorescent molecules distributed in the hydrophobic core do not emit light under certain excitation conditions (for example, in the case of near infrared ray as the excitation light source) due to the FRET effect. The addition of the degradability-regulating group (i.e., B₁ group) can adjust the degradation performance of the polymer in vivo. The delivery molecular group (i.e., the D₁ group) is connected to the main chain of the functionalized diblock copolymer. However, after being administered to an individual, the polymer particles can be enriched at the target site (for example, tumor site) through EPR passive targeting (or other tissue uptake methods). Because the target site has a special pH environment (for example, acidic environment), the protonatable group (i.e., the A₁ group) can be protonated in this pH range, and the charge repulsion generated by its protonation and the increase in polymer solubility drive the disintegration of the polymer particles. The FRET effect of the fluorescent group on the molecular segment is reduced or even completely eliminated. The polymer molecules in the dispersed state enriched in the target site can emit fluorescence under certain excitation conditions (for example, with the near-infrared ray as the excitation light source). In addition to the fluorescent molecular groups carried by the polymer particles, the delivery molecular groups attached to the side chains can continue to be hydrolyzed to the corresponding molecules under specific pH conditions at the target site after polymer dissolution. These molecules can play a corresponding role at the target site. For example, the delivery molecular group can be the group corresponding to 5-ALA, which can provide 5-ALA molecules after hydrolysis. 5-ALA can be efficiently enriched inside cancer cells with accelerated metabolism within a few hours and complete biosynthesis to form Protoporphyrin. At this time, fluoresce can be efficiently emitted under the irradiation of near-infrared excitation. Together with ICG fluorescent molecules, the effect of fluorescence image enhancement or boundary confirmation at the tumor site can be realized. Moreover, 5-ALA is an approved precursor of photodynamic therapy drugs. In this embodiment, we creatively introduce and deliver 5-ALA, which not only enhances the effect of tumor-specific imaging, but also performs photodynamic therapy at tumor sites at the same time. In addition to the fluorescent molecular groups carried by the polymer particles, the insoluble anticancer drugs connected to the side chains can form a good water-soluble, safe and stable pharmaceutical injection preparation. On the one hand, this pharmaceutical preparation greatly increases the solubility of hydrophobic drugs in the blood and reduces their direct contact with the blood, which improves the stability of the drug in the body, reduces the toxic and side effects of the drug, and retains the high anti-tumor activity characteristics of the drugs. After the polymer is disintegrated, the delivery molecular groups attached to the side chains can continue to be hydrolyzed to the corresponding molecules under specific pH conditions at the target site. These molecules can play a corresponding role at the target site. For example, the delivery molecule group can be the group corresponding to SN-38, which can provide SN-38 after hydrolysis, which overcomes the shortcomings of conventional hydrophobic antitumor drug delivery systems such as low drug loading capacity and strong side effects, thus improving drug safety and achieving the effect of killing cancer cells. In addition, the side chains can also be chemically connected to nucleic acid drugs or deliver nucleic acid drugs through physical action, forming a nano-formulation of nucleic acid drugs, which can significantly improve the in vivo stability of nucleic acid drugs. After the polymer particle is disintegrated, the delivery molecular groups attached to the side chains can continue to be hydrolyzed (corresponding to chemical linkage) or released (corresponding to physical interaction delivery) into the corresponding nucleic acid drug molecules under specific pH conditions at the target site to exert the drug efficacy at the site of the lesion.

In a preferred embodiment, the chemical structural formula of the functionalized diblock copolymer is shown as one of the following:

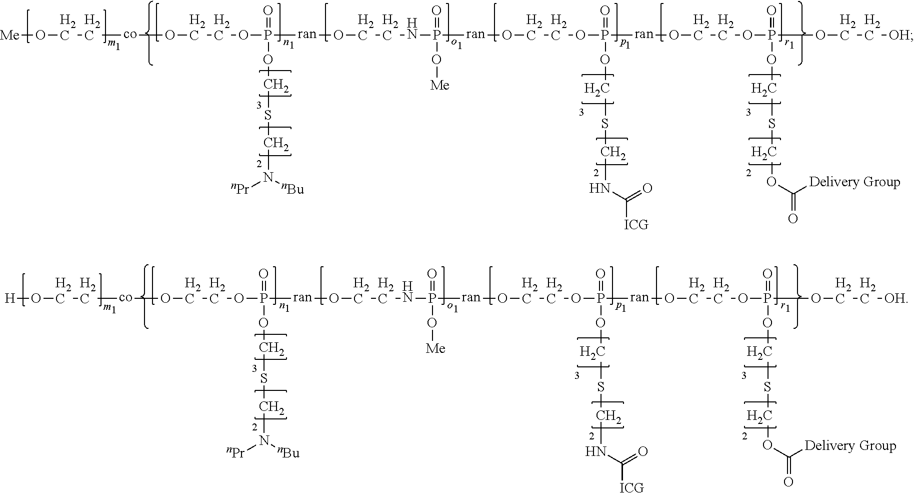

In another preferred embodiment, m₁=44˜226, n₁=50˜300, o₁=1˜10, p₁=0.5˜5, and q₁=10˜300.

The functionalized diblock copolymer provided in the present disclosure usually has a low critical micelle concentration (CMC), thereby reducing the difficulty of preparing polymer self-assembled particles, thereby ensuring that the prepared polymer particles have good stability in solution and blood. For example, the critical micelle concentration (CMC) of the functionalized diblock copolymer may be <50 μg/mL, <45 μg/mL, <40 μg/mL, <35 μg/mL, <30 μg/mL, <25 μg/mL, <20 μg/mL, <16 μg/mL, <14 μg/mL, <12 μg/mL, <10 μg/mL, ≤9 μg/mL, ≤8 μg/mL, ≤7 μg/mL, ≤6 μg/mL, ≤5 μg/mL, ≤4 μg/mL, or smaller critical micelle concentration.

The second aspect of the present disclosure provides polymer particles prepared from the functionalized diblock copolymer provided in the first aspect of the present disclosure. The functionalized diblock copolymers described above can be used to form polymer particles. The fluorescent molecules distributed in the hydrophobic core of the polymer particles do not emit light under certain excitation conditions (for example, in the case of near infrared as the excitation light source) due to the FRET effect. However, after being administered to an individual, the polymer particles can be enriched at the target site (for example, tumor site) through EPR passive targeting (or other tissue uptake methods). Because the target site has a special pH environment (for example, acidic environment), the protonatable group can be protonated in this pH range, and the charge repulsion generated by its protonation and the increase in polymer solubility drive the disintegration of the polymer particles. The FRET effect of the fluorescent group on the molecular segment is reduced or even completely eliminated. The polymer molecules in the dispersed state enriched in the target site can emit fluorescence under certain excitation conditions (for example, with the near-infrared ray as the excitation light source). For example, the PH values in the aforementioned pH environment can be 6.5-6.8, which can correspond to the interstitial fluid of tumor tissue, and at least part of the polymer particles can reach the target site and stay in the interstitial fluid of the cells; for another example, the PH values in the aforementioned pH environment can also be 4.5-6.5, which correspond to endosomes or lysosomes in tumor cells, and at least part of the polymer particles can interact with cells (for example, tumor cells) at the target site and enter into the cells through the endocytosis mechanism, thus reaching the above pH environment. The polymer particles prepared by the functionalized diblock copolymer provided in the present disclosure can be sufficiently diffused at the target site to achieve a clear fluorescence margin, and the functionalized diblock copolymer and/or polymer particles are bio-degradable in vivo. After being administered to an individual, polymer particles or nano-particles that fail to be targeted to the tumor site through the EPR effect cycle may be up-taken by the body's immune system (mainly macrophages, etc.) and then degraded (although PEG cannot be completely degraded in the body, PEG molecules with a molecular weight of less than 40,000 Da (for example, Roche's long-acting interferon, PEGASYS®, has been approved for safe clinical use for more than ten years, and its molecular weight is 40,000 Da) can be effectively eliminated by the kidneys after circulating in the body; PPE can be enzymatically degraded by protein hydrolases, such as phosphodiesterase, and gradually metabolized after the molecular weight gradually decreases, and partly can be cleared by the kidneys). The polymer particles targeted to the target site through the EPR effect are disintegrated into free functionalized diblock copolymer molecules, which, under the pH conditions of the target site and the presence of a variety of enzymes, can be degraded into PEG (which can be cleared by the kidney after circulation) and degradable block (PPE) polymers with gradually smaller molecular weights (which can be subsequently metabolized by circulation and partially cleared by the kidneys). These degradation pathways can improve the safety of the drug system for imaging probe applications or drug delivery system applications that are administered in single or multiple doses. Imaging observation results of live animals show that the block copolymer used can quickly achieve clear fluorescence imaging of tumor tissue after being injected into the living body. After about ten days of follow-up observation, it was found that the fluorescence presented at other sites (liver, kidney, pancreas, etc.) upon injection (this fluorescence appeared in these organs probably because some of the nanoparticles were captured by the reticuloendothelial system (RES) and then phagocytosed by macrophages and other cells, after that, the nanoparticles were protonated and finally disassembled into individual polymer chain segments) almost completely disappeared, providing strong evidence of the biodegradation and clearance performance of the present disclosure.

The polymer particles provided in the present disclosure can be nano-sized. For example, the particle size of the polymer particles can be 10˜200 nm, 10˜20 nm, 20˜30 nm, 30˜40 nm, 40˜60 nm, 60˜80 nm, 80 nm˜100 nm, 100˜120 nm, 120˜140 nm, 140˜160 nm, 160˜180 nm, or 180˜200 nm.

In the polymer particles provided in the present disclosure, the polymer particles can also be modified with targeting groups, and these targeting groups can usually be modified on the surface of the polymer particles. Suitable methods for modifying the targeting group on the polymer particles should be known to those skilled in the art. For example, in general, the targeting group can be attached to the T end of the molecular structure of the functionalized diblock copolymer. These targeting groups can usually increase the efficiency of targeting nanoparticles to liver tumors based on the EPR effect. These targeting groups can include but are not limited to various functional molecules such as (monoclonal) antibody fragments (for example, Fab, etc.), small molecule targeting groups (for example, folic acid, carbohydrates), polypeptide molecules (for example, cRGD, GL2P), and aptamers, and these functional molecules may have a targeting function (for example, a function of targeting tumor tissue). In a specific embodiment of the present disclosure, the targeting group is selected from -GalNac (N-acetylgalactosamine).

The third aspect of the present disclosure provides a method for preparing the polymer particles provided in the second aspect of the present disclosure. Based on the knowledge of the chemical structure of the functionalized diblock copolymer, a suitable method for forming polymer particles shall be known to those skilled in the art. For example, the method may include: dispersing an organic solvent including the above-mentioned functionalized diblock copolymer in water for self-assembling to provide the polymer particles; or conversely, dispersing water in an organic solvent including the functionalized diblock copolymer. In the above dispersion process, proper operations can be used to make the system fully mixed, for example, it can be carried out under ultrasonic conditions. For another example, the self-assembly process can usually be carried out by removing the organic solvent in the reaction system. The organic solvent removal method may specifically be a solvent volatilization method, an ultrafiltration method, and the like. For another example, the critical micelle concentration (CMC) of a polymer is related to the ratio of the hydrophobic block to the hydrophilic block of the polymer. The higher the ratio of the hydrophobic block, the smaller the CMC. When E₁, E₂, E₃ are long-chain hydrophobic side chains, their content is inversely proportional to the value of CMC; when E₁, E₂, E₃ are hydrophilic side chains, their content is directly proportional to the value of CMC. For another example, the particle size of polymer particles can usually be adjusted by an extrusion instrument (NanoAssemblr).

The fourth aspect of the present disclosure provides the uses of the functionalized diblock copolymer provided in the first aspect of the present disclosure or the polymer particles provided in the second aspect of the present disclosure in the preparation of pharmaceutical preparations and/or reagents. Polymer nanoparticles formed as a drug delivery system allow the delivery of drugs or imaging probe molecules using polymer particles as carriers. As mentioned above, the products (for example, polymer particles) prepared by the functionalized diblock copolymers provided in the present disclosure have a passive targeting (enriched at the tumor sites through the general EPR effect of nanoparticles) or active targeting (enriched at the tumor sites by specific binding of nanoparticle surface-modified targeting groups to tumor surface-specific receptors) function. After administration to the individual, because the target site has a special pH environment (for example, acidic environment), the protonatable group can be protonated in this pH range. The charge repulsion generated by the protonation and the increase in polymer solubility drive the disintegration of polymer particles, and the FRET effect of the fluorophore on the dispersed single polymer segment is reduced or even completely eliminated, allowing the polymer molecules in the dispersed state enriched in the target site to emit fluorescence under certain excitation conditions (for example, in the case of near-infrared ray as the excitation light source). In this case, the target site (for example, the tumor site) can emit light specifically, which can be used for targeted imaging probing. In addition to imaging probe applications, these polymer particles can be used to prepare targeting agents. In a specific embodiment of the present disclosure, the above polymer particles can be used to prepare polymer particle-based drug delivery systems to deliver various drug molecules.

In the pharmaceutical preparations or reagents provided by the present disclosure, polymer particles can usually be used as carriers to deliver drugs or imaging probe molecules. The functionalized diblock copolymer can be used as a single active ingredient or can be combined with other active components to collectively form the active ingredient for the aforementioned uses.

The fifth aspect of the present disclosure provides a composition including the functionalized diblock copolymer provided in the first aspect of the present disclosure or the polymer particles provided in the second aspect of the present disclosure. As mentioned above, the aforementioned composition may be a targeting agent, and in a specific embodiment of the present disclosure, the aforementioned composition may be an imaging probe.

The composition provided in the present disclosure may also include at least one pharmaceutically acceptable carrier, which usually refers to a carrier for administration, which does not induce the production of antibodies harmful to the individual receiving the composition, and are not excessively toxic after administration. These carriers are well-known to those skilled in the art. For example, Remington's Pharmaceutical Sciences (Mack Pub. Co., N. J. 1991) discloses related content about pharmaceutically acceptable carriers. Specifically, the carrier may include one or more of saline, buffer, glucose, water, glycerol, ethanol, and adjuvant.

In the composition provided in the present disclosure, the functionalized diblock copolymer may serve as a single active ingredient, or may be combined with other active components for joint use. The other active components can be various other drugs and/or agents, which can usually act on the target site together with the above-mentioned functionalized diblock copolymer. The content of the active ingredient in the composition is usually a safe and effective amount, and the safe and effective amount should be adjustable for those skilled in the art. For example, the dosage of the active ingredient usually depends on the body weight of the subject to whom it is administered, the type of application, and the condition and severity of the disease.

The composition provided in the present disclosure can be adapted to any form of administration. It can be parenterally administered, for example, it can be pulmonary, nasal, rectal and/or intravenous injection, more specifically intradermal, subcutaneous, intramuscular, intraarticular, intraperitoneal, lung, oral, sublingual, nasal, percutaneous, vaginal, bladder instillation, uterine perfusion, intestinal perfusion, topical administration after craniotomy, or parenteral administration. Those skilled in the art can choose a suitable preparation form according to the mode of administration. For example, the preparation form suitable for parenteral administration may include, but is not limited to, a solution, a suspension, a reconstitutable dry preparation or a spray, etc. For another example, it may be in the form of a preparation administered by inhalation in the form of an inhalant.

The sixth aspect of the present disclosure provides a method of treatment or diagnosis, including: administering to an individual an effective amount of the functionalized diblock copolymer provided in the first aspect of the present disclosure, or the polymer particles provided in the second aspect of the present disclosure, or the composition provided by the fifth aspect of the present present disclosure. The “effective amount” generally refers to an amount that can achieve the desired effect after a proper administration period, for example, imaging, treatment of diseases, etc. The above-mentioned are pH-responsive and can be degraded under corresponding pH conditions. Chemical modifications on the functionalized diblock copolymers can also bring synergistic effect from co-delivered molecules, which are bonded to polymer molecules through degradable chemical bonds, and can be combined with a unique end group (targeting group, a group that can improve the immunogenicity of the system) to become a unique block copolymer-delivery bonded complex. In a specific embodiment of the present disclosure, after use, better intraoperative tumor boundary discrimination, and more precise removal of tumor lesions and metastatic tissue can be achieved. During the intraoperative imaging, the local delivery molecules can be used to better kill cancer cells, reduce the recurrence rate, and improve the patient's postoperative survival.

The functionalized diblock copolymers or polymer particles provided in the present disclosure can significantly improve the safety of tumor imaging probe reagents and/or tumor drug preparations (tumor imaging probe reagents are mostly for single use; and tumor drugs are usually administered multiple times). For the diblock copolymer provided by the present disclosure (the compound of formula I, or PEG-PPE diblock copolymer), PEG can be safely removed from the human body (ADEGEN®, ONCASPAR®, etc., are clinically approved to use PEG with a molecular weight of 5K in multi-site modified therapeutic enzymes; biological macromolecules such as interferon, granulocyte colony stimulating factor, and antibody Fab fragments modified with 12-40K PEG have been safely used in clinical practice for more than ten years), another block PPE can be gradually degraded under physiological conditions (hydrolysis; enzyme). In addition, the design in which the PPE main chain can be actively severed under acidic conditions allows for faster and adjustable (by changing the number of functional groups) degradation and removal of the polymer under acidic conditions.

The functionalized diblock copolymers or polymer particles provided in the present disclosure can achieve high-quality imaging with tumor imaging probe reagents specific to solid tumor sites, and can be sensitive to pH changes at the tumor site (fluorescence signal changes ΔpH10-90% only need about 0.2-0.3 pH unit), with high signal-to-noise ratio, clear boundary, and long half-life. Live imaging data show that the imaging probe used can have a long intratumoral retention and duration (several days or more) once enriched into the tumor, giving a longer observation window for tumor imaging surgery, and solving the problem of fluorescence imaging technology in real-time intraoperative navigation.

The functionalized diblock copolymers, polymer particles, or compositions provided in the present disclosure can be conveniently administered locally, for example, bladder instillation, uterine perfusion, intestinal perfusion, local administration to the brain after craniotomy. The polymer particles used can be absorbed by the tumor tissue after sufficient contact with the tumor tissue, thereby achieving imaging and treatment of the tumor tissue.

The functionalized diblock copolymers or polymer particles provided in the present disclosure can introduce, based on the feature that the nanoparticles can accumulate sufficiently into the solid tumor microenvironment, precursor molecules (e.g., precursor molecules for photodynamic therapeutics, more specifically precursor molecules of 5-ALA) to the polymers, where these precursor molecules can be cleaved and released to the tumor microenvironment (weak acids, tumor microenvironment-specific proteases, etc.). The side chains can be cleaved from the polymer backbone and converted to the clinically approved drug molecules (e.g., 5-ALA, etc.), enabling intraoperative image enhancement of tumor sites. At the same time as the implementation of imaging, the designed imaging probe reagent utilizes the light source of intraoperative imaging to realize the photodynamic therapy of tumor tissue during tumor resection surgery, and reduce the damage of other photodynamic therapy on normal tissue, kill the uncut cancer tissue in the process of resection of the tumor tissue, reduce postoperative recurrence and prolong survival time.

In summary, the functionalized diblock copolymers or polymer particles provided in the present disclosure can be widely used in tumor imaging, tumor therapy and other fields. It not only has good safety, realizes faster and adjustable degradation and removal of polymers (by changing the structure and number of functional groups) under acidic conditions, but also has excellent specific and high-quality imaging effects at the target site, with high signal-to-noise ratio, clear boundaries, long half-life, etc., which solves the problem of fluorescence imaging technology in real-time intraoperative navigation, and thus has a good industrialization prospect.

The following embodiments further illustrate the present disclosure, but do not limit the scope of the present disclosure.

The reaction route of the preparation method of the compound of formula I series in the embodiment is as follows:

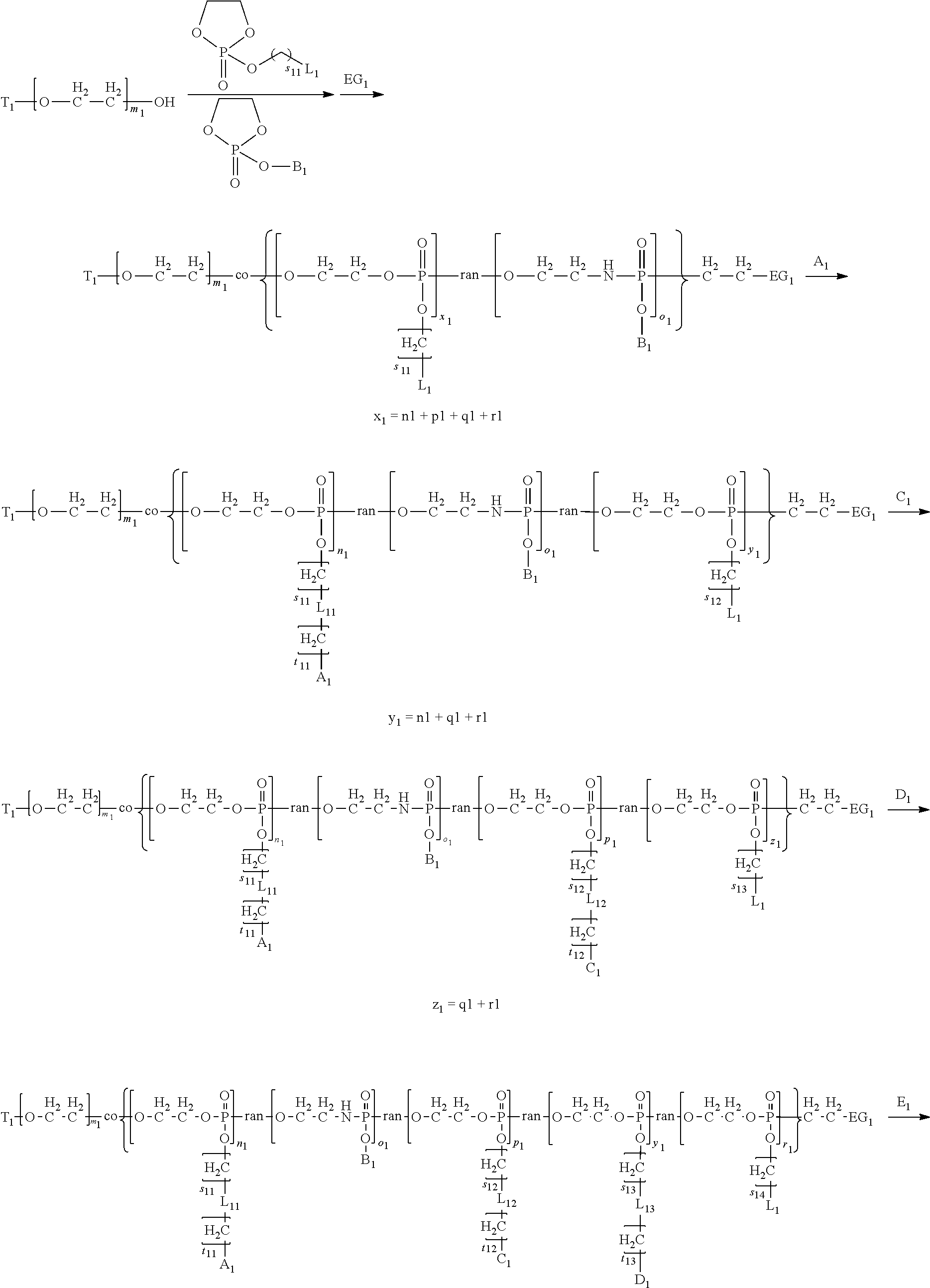

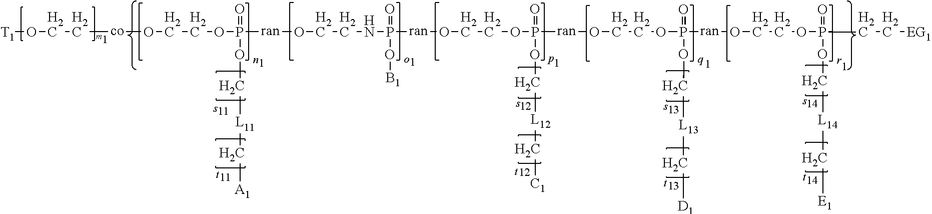

### EXAMPLE 1

### Synthesis of mPEG-PPE Copolymer

### 1.1 Synthesis of Monomer:

### Synthesis of AEP (IB001-077-01):

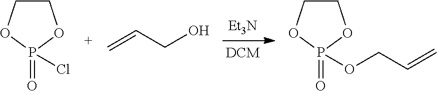

Allyl alcohol (11.6 g, 0.2 mol) was dissolved in 250 ml of dry DCM, dry triethylamine (20.2 g, 0.2 mol) was added, and then the mixture was cooled to 0° C. in an ice-salt bath. Argon displacement was performed three times.

2-Chloro-2-oxo-1,3,2-dioxaphospholane (28.4 g, 0.2 mol) was added slowly dropwise to the above reaction solution, and the temperature was kept below 5° C. After the dropwise adding, the reaction system continued to be stirred at 0° C. for 3 h. Most of the DCM was concentrated off, then 200 ml of dry methyl tert-butyl ether was added. A white solid was precipitated, filtered, washed with 20 ml of methyl tert-butyl ether, and the filtrate was concentrated. Finally, the obtained concentrate was distilled under reduced pressure (0.1 torr, 92° C.) to give 13.7 g of product, which was a colorless and transparent liquid with a yield of 41.7%. The product was stored at −20° C. ¹H NMR (400 MHz, CDCl₃) δ 5.97 (ddt, J=16.4, 10.9, 5.7 Hz, 1H), 5.46-5.36 (m, 1H), 5.29 (dd, J=10.4, 1.4 Hz, 1H), 4.69-4.58 (m, 2H), 4.50-4.33 (m, 4H).

### 1.2 Polymerization:

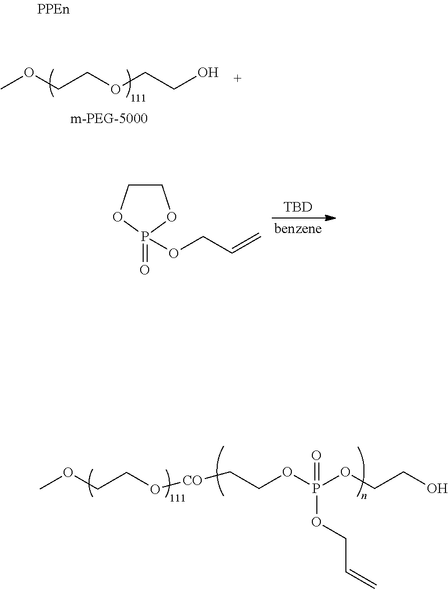

### 1.2.1 General Polymerization Method, PPE70(n=70, IB004-030-01):

In a glove box with H₂O and O₂ indexes less than 0.1 ppm, mPEG-5000 (100 mg, 0.02 mmol) was place in a polymerization reaction tube, and 0.5 ml of benzene was added. Then, the polymerization reaction tube was sealed and removed from the glove box, heated to 50° C., stirred for 10 min. After the reagents were all dissolved, the tube was cooled down to room temperature, and then moved back into the glove box. AEP (328 mg, 2 mmol) was added. Finally, TBD (2.78 mg, 0.02 mmol) was added, and the mixture was rapidly stirred to react for 5 min. The polymerization reaction tube was removed from the glove box, the reaction was terminated by adding benzoic acid solution (30 mg of benzoic acid dissolved in 1 ml of DCM). The reaction was stirred for 5 min, then 50 ml of methyl tert-butyl ether was added slowly. A white precipitate appeared. The stirring was continued for 10 min, and filtration was performed to obtain 268 mg of white solid polymer with a yield of 78.2%. ¹H NMR (400 MHz, CDCl₃) δ 6.00-5.90 (m, 70H), 5.36 (d, J=17.1, 1.7 Hz, 70H), 5.27 (d, J=10.6, 1.5 Hz, 70H), 4.58 (dd, J=8.1, 5.8 Hz, 140H), 4.32-4.20 (m, 280H), 3.64 (s, 448H), 3.38 (s, 3H). Mw: 18045, Mn: 12600, PDI: 1.432.

1.2.2 PPE90 (n=90)

Synthesis and purification of PPE90 was carried out according to the process as in Example 1.2.1 above, to obtain 386 mg of white solid polymer in 90.2% yield. ¹H NMR (400 MHz, CDCl₃) δ 5.99-5.90 (m, 90H), 5.37 (d, J=17.1, 1.7 Hz, 90H), 5.26 (d, J=10.6, 1.5 Hz, 90H), 4.54 (dd, J=8.1, 5.8 Hz, 180H), 4.30-4.20 (m, 360H), 3.64 (s, 448H), 3.38 (s, 3H). Mw: 18945, Mn: 14127, PDI: 1.341.

1.2.3 PPE120 (n=120)

Synthesis and purification of PPE120 was carried out according to the process as in Example 1.2.1 above, to obtain 479 mg of white solid polymer in 89.8% yield. ¹H NMR (400 MHz, CDCl₃) δ 5.99-5.91 (m, 123H), 5.35 (d, J=17.1, 1.7 Hz, 123H), 5.25 (d, J=10.6, 1.5 Hz, 123H), 4.54 (dd, J=8.1, 5.8 Hz, 246H), 4.28-4.24 (m, 492H), 3.64 (s, 448H), 3.37 (s, 3H). Mw: 21479, Mn: 14461, PDI: 1.485.

1.2.4 PPE150 (n=150)

Synthesis and purification of PPE150 was carried out according to the process as in Example 1.2.1 above, to obtain 577 mg of white solid polymer in 95.3% yield. ¹H NMR (400 MHz, CDCl₃) δ 5.99-5.90 (m, 146H), 5.36 (d, J=17.1, 1.7 Hz, 146H), 5.26 (d, J=10.6, 1.5 Hz, 146H), 4.54 (dd, J=8.1, 5.8 Hz, 292H), 4.27-4.24 (m, 584H), 3.64 (s, 448H), 3.38 (s, 3H). Mw: 33489, Mn: 22443, PDI: 1.492.

1.2.5 PPE200 (n=200)

Synthesis and purification of PPE200 was carried out according to the process as in Example 1.2.1 above, to obtain 638 mg of white solid polymer in 92.4% yield. ¹H NMR (400 MHz, CDCl₃) δ 5.94 (ddt, J=16.4, 10.9, 5.7 Hz, 87H), 5.38 (dd, J=17.1, 1.6 Hz, 89H), 5.27 (dd, J=10.5, 1.4 Hz, 88H), 4.58 (dd, J=8.1, 5.9 Hz, 180H), 4.36-4.17 (m, 367H), 3.64 (s, 448H).3.38(s, 3H). Mw: 39356, Mn: 21908, PDI: 1.796.

1.2.6 PPE250 (n=250)

Synthesis and purification of PPE250 was carried out according to the process as in Example 1.2.1 above, to obtain 769 mg of white solid polymer in 93.6% yield. ¹H NMR (400 MHz, CDCl₃) δ 5.99-5.91 (m, 258H), 5.36 (d, J=17.1, 1.7 Hz, 258H), 5.26 (d, J=10.6, 1.5 Hz, 258H), 4.54 (dd, J=8.1, 5.8 Hz, 516H), 4.28-4.25 (m, 1032H), 3.64 (s, 448H), 3.38 (s, 3H). Mw: 39902, Mn: 22993, PDI: 1.735.

1.2.7 PPE300 (n=300)

Synthesis and purification of PPE300 was carried out according to the process as in Example 1.2.1 above, to obtain 1048 mg of white solid polymer in 97.0% yield. ¹H NMR (400 MHz, CDCl₃) δ 5.99-5.91 (m, 290H), 5.36 (d, J=17.1, 1.7 Hz, 290H), 5.26 (d, J=10.6, 1.5 Hz, 290H), 4.54 (dd, J=8.1, 5.8 Hz, 580H), 4.27-4.25 (m, 1160H), 3.65 (s, 448H), 3.38 (s, 3H). Mw: 43351, Mn: 24337, PDI: 1.781.

1.2.8 HO-PPE90 (n=90)

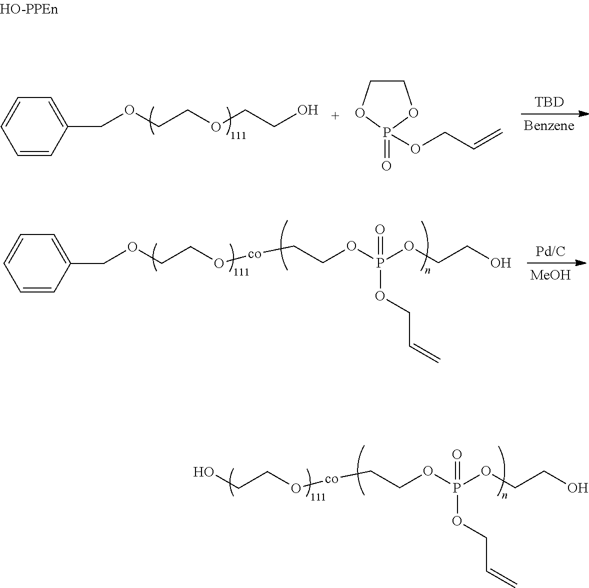

### Step One, Bn-PPE90:

Synthesis and purification of Bn-PPE90 was carried out according to the process as in Example 1.2.1 above (m-PEG-5000 was replaced with an equimolar amount of Bn-PEG-5000), to obtain 605 mg of white solid polymer in 92.1% yield. ¹H NMR (400 MHz, CDCl₃) δ 7.30 (m, 5H), 5.99-5.90 (m, 90H), 5.37 (d, J=17.1, 1.7 Hz, 90H), 5.26 (d, J=10.6, 1.5 Hz, 90H), 4.58 (dd, J=8.1, 5.8 Hz, 182H), 4.31-4.22 (m, 360H), 3.66 (s, 448H). Mw: 19744, Mn: 15217, PDI: 1.297.

### Step Two, OH-PPE90

In a 25 mL high-pressure reactor, 500 mg of Bn-PPE90 was added, fully dissolved in 5 mL of methanol, then 50 mg of Pd/C was added. The reactor was pressurized to 500 PSI, and the temperature was raised to 50° C. After 48 h, the reaction was stopped and filtration was performed, and the filtrate was slowly added with 50 ml of methyl tertiary-butyl ether, which resulted in the appearance of a white precipitate. The reaction was stirred for 10 min, and filtration was performed to obtain 370 mg of white solid polymer with a yield of 74.4%. ¹H NMR (400 MHz, CDCl₃) δ 5.99-5.89 (m, 90H), 5.38 (d, J=17.1, 1.7 Hz, 90H), 5.26 (d, J=10.6, 1.5 Hz, 90H), 4.55 (dd, J=8.1, 5.8 Hz, 182H), 4.30-4.19 (m, 360H), 3.64 (s, 450H), 3.38. Mw: 19046, Mn: 14088, PDI: 1.352.

### 1.3 Synthesis of Side Chains:

### 1.3.1 Synthesis of TEPr:

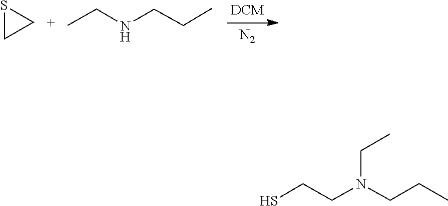

N-ethyl-n-propylamine (34.8 g, 0.4 mol) and 500 ml of dichloromethane were added to a 1 L three-necked flask sequentially and the system was replaced with N₂ three times, then thiirane (48 g, 0.8 mol) was added slowly dropwise to the above solution. After completion of the dropwise adding, the system was stirred and reacted overnight at room temperature. The reaction was terminated, and the organic solvent was removed by concentration. Finally, the obtained concentrate was distilled under reduced pressure (0.2 torr, 38° C.) to obtain 24 g of product, which was a colorless and transparent liquid with a yield of 40.8%. ¹H NMR (400 MHz, CDCl₃) δ 4.81 (d, J=8.5 Hz, 4H), 2.67-2.46 (m, 6H), 2.37 (dd, J=8.6, 6.5 Hz, 2H), 1.51-1.37 (m, 2H), 1.00 (t, J=7.1 Hz, 3H), 0.87 (t, J=7.3 Hz, 3H).

### 1.3.2 Synthesis of TPrPr:

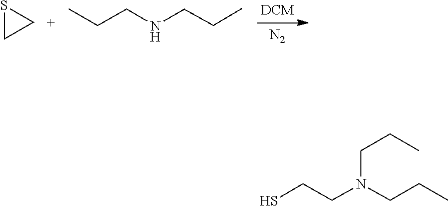

Di-n-propylamine (40.4 g, 0.4 mol) and 500 ml of dichloromethane were added to a 1 L three-necked flask sequentially and the system was replaced with N₂ three times, then thiirane (48 g, 0.8 mol) was added slowly dropwise to the above solution. After completion of the dropwise adding, the system was stirred and reacted overnight at room temperature. The reaction was terminated, and the organic solvent was removed by concentration. Finally, the obtained concentrate was distilled under reduced pressure (0.2 torr, 42° C.) to obtain 21 g of product, which was a colorless and transparent liquid with a yield of 32.6%. ¹H NMR (400 MHz, CDCl₃) δ 2.69-2.54 (m, 4H), 2.39 (dd, J=8.5, 6.6 Hz, 4H), 1.46 (h, J=7.4 Hz, 4H), 0.89 (t, J=7.4 Hz, 6H).

### 1.3.3 Synthesis of TPrB:

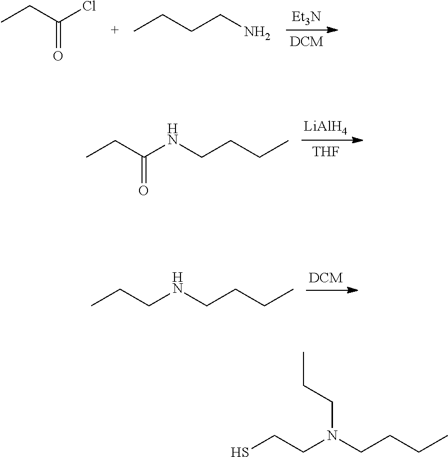

### Step 1: Synthesis of n-butyl Propionamide (IB001-183-01)

N-butylamine (40.15 g, 0.55 mol) and Et₃N(101 g, 1 mol) were dissolved in 500 ml DCM, cooled to 0° C. in ice bath, and subjected to nitrogen replacement three times. Propionyl chloride (46.25 g, 0.5 mol) was slowly dropped into the above solution. After dropping, the solution was stirred at room temperature overnight. The salt of Et 3 N was removed by filtration, the solvent was removed by concentration, and the crude product was distilled under reduced pressure (80° C./0.4 torr) to obtain 45 g of product, which was a colorless and transparent liquid with a yield of 69.7%.

### Step 2: Synthesis of Butyl Propylamine (IB001-186-01)

N-butyl propionamide (38.7 g, 0.3 mol) was dissolved in 500 ml THF, and LiAlH₄(12.54 g, 0.33 mol) was added in batches with stirring, and the reaction was refluxed overnight. The reaction solution was cooled, and was slowly added with 98 ml of 1 mol/L NaOH solution under stirring, then, the solution was filtered through diatomaceous earth, and the filtrate was concentrated, then extracted by EA (50 ml×3). The organic phases were combined, washed with H₂O (50 ml×1), NaCl (50 ml×1), respectively, and dried with anhydrous Na₂SO₄, then filtered, and concentrated to obtain crude product. The crude product was distilled under reduced pressure (65° C./0.4 torr) to obtain 12.5 g of butyl propylamine, which was a colorless transparent liquid with a yield of 36.2%.

### Step 1: Synthesis of 2-(butylpropylamino)-ethanethiol (IB001-190-01)

Butyl propylamine (11.5 g, 0.1 mol) was dissolved in 100 ml DCM, and subjected to nitrogen replacement three times. Then, thiirane (12 g, 0.2 mol) was added slowly dropwise to the above solution. After completion of the dropwise adding, the system was stirred and reacted overnight at room temperature. The solvent was removed by concentration, and the crude product was distilled under reduced pressure (73° C./0.4 torr) to obtain 2-(butylpropylamino)-ethanethiol, which was a colorless and transparent liquid with a yield of 34.2%. ¹H NMR (400 MHz, CDCl₃) δ 2.69-2.54 (m, 4H), 2.46-2.39 (dd, J=8.2, 6.6 Hz, 4H), 1.63-1.39 (m, 4H), 1.34 (h, J=7.4 Hz, 2H), 0.91-0.85 (m, 6H).

### 1.3.4 Synthesis of TBB:

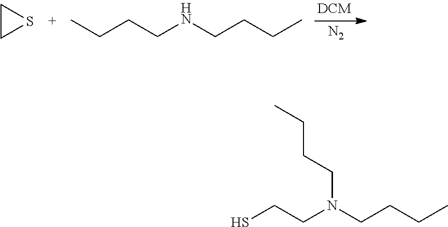

Di-n-butylamine (25.8 g, 0.2 mol) and 300 ml of dichloromethane were added to a 500 mL three-necked flask sequentially and the system was subjected to nitrogen replacement three times, then thiirane (24 g, 0.4 mol) was added slowly dropwise to the above solution. After completion of the dropwise adding, the system was stirred and reacted overnight at room temperature. The reaction was terminated, and the organic solvent was removed by concentration. Finally, the obtained concentrate was distilled under reduced pressure (0.2 torr, 49° C.) to obtain 11.4 g of product, which was a colorless and transparent liquid with a yield of 30.1%. ¹H NMR (400 MHz, CDCl₃) δ 2.63 (dd, J=16.1, 6.2 Hz, 4H), 2.45 (t, J=7.4 Hz, 4H), 1.45 (p, J=7.3 Hz, 4H), 1.34 (p, J=7.2 Hz, 4H), 0.93 (t, J=7.2 Hz, 6H).

### 1.3.5 Synthesis of TBPe:

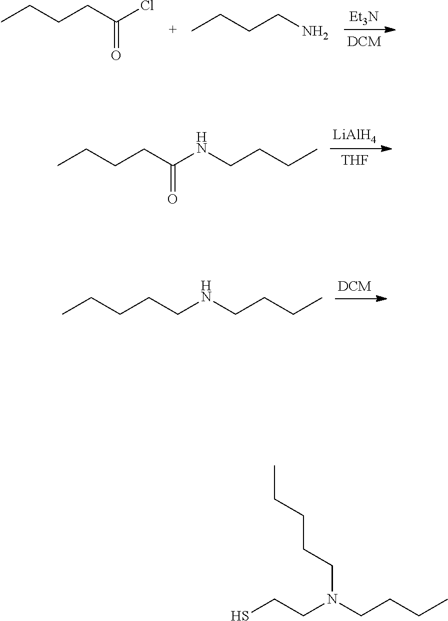

### Step 1: Synthesis of Butyramide Valerate (IB001-176-01)

N-butylamine (16.06 g, 0.22 mol) and Et₃N(40.4 g, 0.4 mol) were dissolved in 2800 ml DCM, cooled to 0° C. in ice bath, and subjected to nitrogen replacement three times. Valeryl chloride (24 g, 0.2 mol) was slowly dropped into the above solution. After dropping, the solution was stirred at room temperature overnight.

The salt of Et₃N was removed by filtration, the solvent was removed by concentration, and the crude product was distilled under reduced pressure (82° C./0.4 torr) to obtain 16.3 g of product, which was a colorless and transparent liquid with a yield of 52%. ¹H NMR (400 MHz, CDCl₃) δ 3.24 (td, J=7.2, 5.7 Hz, 2H), 2.16 (t, J=7.7 Hz, 2H), 1.61 (dq, J=8.9, 7.5 Hz, 2H), 1.55-1.42 (m, 2H), 1.34 (h, J=7.3 Hz, 4H), 0.92 (td, J=7.3, 3.0 Hz, 6H).

### Step 2: Synthesis of Butyl Amylamine (IB001-179-01)

Butyramide valerate (15.7 g, 0.1 mol) was dissolved in 200 ml THF, and LiAlH₄ (4.18 g, 0.11 mol) was added in batches under stirring, and the reaction was refluxed overnight. The reaction solution was cooled, and was slowly added with 98 ml of 1 mol/L NaOH solution under stirring, then the solution was filtered through diatomaceous earth, and the filtrate was concentrated, then extracted by EA (50 ml×3). The organic phases were combined, washed with H₂O (50 ml×1), NaCl (50 ml×1), respectively, and dried with anhydrous Na₂SO₄, then filtered, and concentrated to obtain crude product. The crude product was distilled under reduced pressure (68° C./0.4 torr) to obtain 5.4 g of butyl amylamine, which was a colorless transparent liquid with a yield of 38%. ¹H NMR (400 MHz, CDCl₃) δ 62.57 (m, 4H), 1.25-1.55(m, 11H), 0.89 (m, 6H).

### Step 3: Synthesis of 2-(butylpentylamino)-ethanethiol (IB001-180-01)

Butyl amylamine (10 g, 0.07 mol) was dissolved in 50 ml DCM, and subjected to nitrogen replacement three times. Then, thiirane (8.4 g, 0.14 mol) was added slowly dropwise to the above solution. After completion of the dropwise adding, the system was stirred and reacted overnight at room temperature. The solvent was removed by concentration, and the crude product was distilled under reduced pressure (76° C./0.4 torr) to obtain 4.4 g 2-(butylpentylamino)-ethanethiol, which was a colorless and transparent liquid with a yield of 31%. ¹H NMR (400 MHz, CDCl₃) δ 2.70-2.53 (m, 4H), 2.42 (td, J=7.5, 3.5 Hz, 4H), 1.52-1.20 (m, 10H), 0.92 (q, J=7.1 Hz, 6H).

### 1.3.6 Synthesis of TPePe (IB001-172-01):

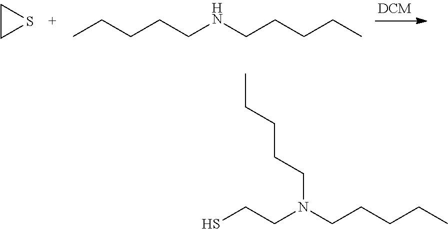

Diamylamine (15.7 g, 0.1 mol) was dissolved in 200 ml DCM, and subjected to nitrogen replacement three times. Then, thiirane (12 g, 0.2 mol) was added slowly dropwise to the above solution. After completion of the dropwise adding, the system was stirred and reacted overnight at room temperature. The solvent was removed by concentration, and the crude product was distilled under reduced pressure (83° C./0.4 torr) to obtain 9.1 g 2-(dipentylamino)-ethanethiol, which was a colorless and transparent liquid with a yield of 42%.

¹H NMR (400 MHz CDCl₃) δ 2.68-2.54 (m, 4H), 2.49-2.36 (m, 4H), 1.44 (p, J=7.3 Hz, 4H), 1.39-1.20 (m, 8H), 0.91 (t, J=7.0 Hz, 6H).

### 1.3.7 Synthesis of THH (IB001-172-01):

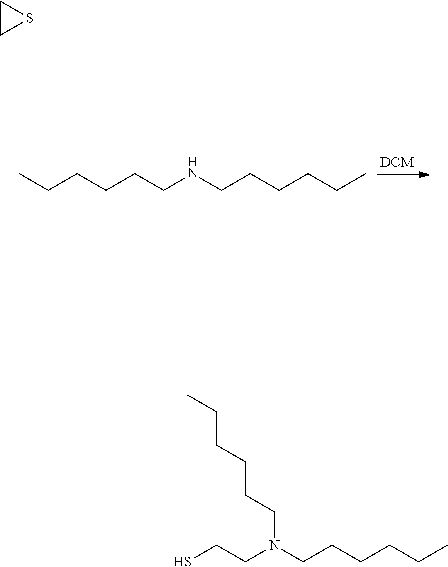

Diamylamine (15.7 g, 0.1 mol) was dissolved in 200 ml DCM, and subjected to nitrogen replacement three times. Then, thiirane (12 g, 0.2 mol) was added slowly dropwise to the above solution. After completion of the dropwise adding, the system was stirred and reacted overnight at room temperature. The solvent was removed by concentration, and the crude product was distilled under reduced pressure (83° C./0.4 torr) to obtain 9.1 g 2-(dipentylamino)-ethanethiol, which was a colorless and transparent liquid with a yield of 42%.

¹H NMR (400 MHz, CDCl₃) δ 2.68-2.54 (m, 4H), 2.49-2.36 (m, 4H), 1.44 (p, J=7.3 Hz, 4H), 1.39-1.20 (m, 8H), 0.91 (t, J=7.0 Hz, 6H).

### 1.3.8 Synthesis of 5-ALA Side Chain

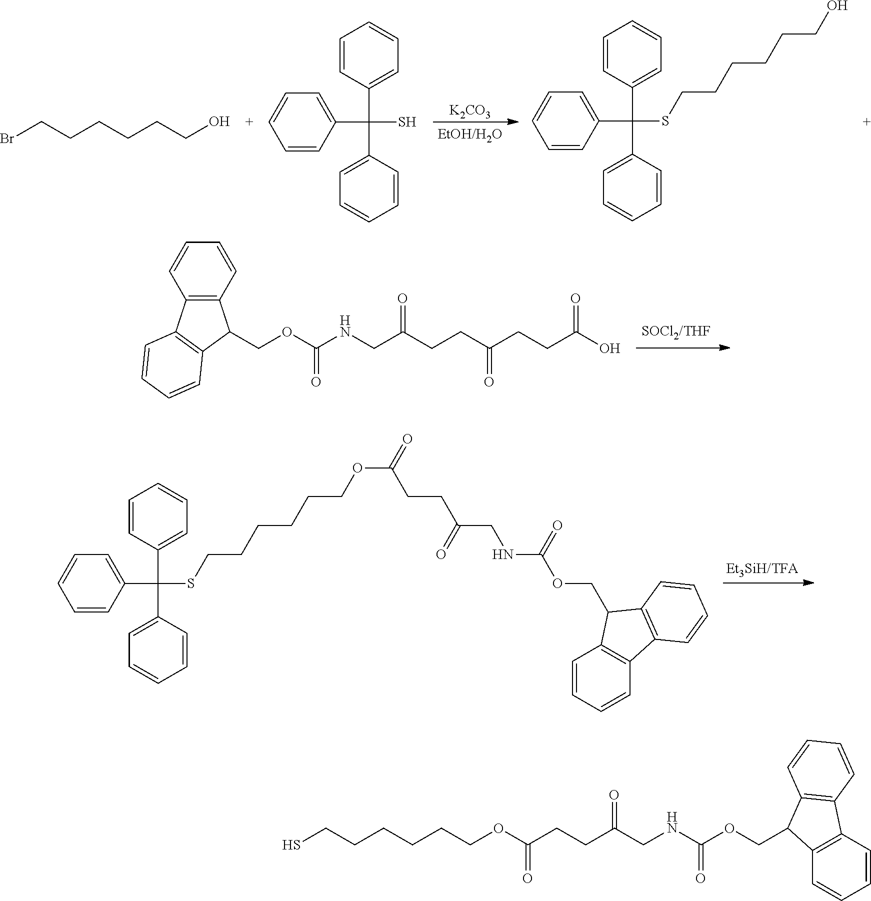

### Step 1: Synthesis of 6-triphenylmercaptohexan-1-ol (IB004-045-01)

Triphenylmethyl mercaptan (8.29 g, 0.03 mol) was dissolved in 30 ml EtOH and 30 ml water, then K₂CO₃ (4.14 g, 0.03 mol) was added. The mixture was stirred for 30 min at room temperature under argon protection, then bromohexanol (5.43 g, 0.03 mol) was added, and the temperature was raised to 80° C. and the mixture was stirred for reaction overnight. The reaction was terminated, and the mixture was filtered and concentrated to remove EtOH. 50 ml of water was added, EA extraction (50 ml×3) was performed, the organic phases were combined, washed with water (50 ml×1), washed with saturated NaCl (50 ml×1), dried with anhydrous Na₂SO₄, filtered, concentrated, and dried by an oil pump to obtain 10.92 g of white solid in 96.4% yield, which was not further purified and will be used directly in the next step of the reaction. ¹H-NMR (500 MHz, Chloroform-d) δ 7.49-7.39 (m, 6H), 7.29 (t, J=7.7 Hz, 6H), 7.25-7.18 (m, 3H), 3.58 (t, J=6.6 Hz, 2H), 2.16 (t, J=7.3 Hz, 2H), 1.54 (s, 1H), 1.53-1.45 (m, 2H), 1.45-1.38 (m, 2H), 1.34-1.18 (m, 4H).

### Step 2: Synthesis of 6-triphenylmercaptohexyl 5-Fmoc-5-amino-4-oxopentanoate (IB004-055-01)

6-Triphenylmercaptohexan-1-ol (3.77 g, 0.01 mol) was dissolved in 30 ml THF, then SOCl₂ (1.67 g, 0.014 mol) was added. The mixture was stirred for 10 min, then 5-Fmoc-5-aminolevulinic acid hydrochloride (1.67 g, 0.01 mol) was added, and the system was stirred and reacted overnight at room temperature. 50 ml saturated NaHCO₃ solution was added slowly, the resulting solution was subjected to EA extraction (50 ml×3), the organic phases were combined, washed with water (50 ml×1), washed with saturated NaCI (50 ml×1), dried with anhydrous Na₂SO₄, filtered, and concentrated. The crude product was separated and purified on silica gel column (EA:PE=1:25), and a total of 4.17 g of product was obtained, which was colorless and transparent oil in nature with 58.7% yield. ¹H-NMR (500 MHz, Chloroform-d) δ 7.89 (m, 2H), 7.73-7.65 (m, 4H), 7.49-7.39 (m, 8H), 7.29 (t, J=7.7 Hz, 6H), 7.25-7.18 (m, 3H), 4.07 (2H, br s), 3.58 (t, J=6.6 Hz, 2H), 2.87 (2H, t, J=6.5 Hz),2.63 (2H, t, J=6.5 Hz), 2.16 (t, J=7.3 Hz, 2H), 1.53-1.45 (m, 2H), 1.45-1.38 (m, 2H), 1.34-1.18 (m, 4H).

### Step 3: Synthesis of 6-mercaptohexyl 5-Fmoc-5-amino-4-oxopentanoate (IB004-063-01)

6-triphenylmercaptohexyl 5-Fmoc-5-amino-4-oxopentanoate (3.56 g, 5 mmol) was dissolved in 50 ml DCM, then Et₃SiH (3.41 g, 29.4 mmol) and TFA (6.7 g, 58.8 mmol) were sequentially added, and the mixture was stirred at room temperature for 1 h. The solvent was removed by concentration, 50 ml of water was added, 50 ml of saturated NaHCO₃ solution was added slowly, and the resulting solution was subjected to EA extraction (50 ml×3). The organic phases were combined, washed with water (50 ml×1), washed with saturated NaCI (50 ml×1), dried with anhydrous Na₂SO₄, filtered, and concentrated. The crude product was separated and purified on silica gel column (EA:PE=1:5), and a total of 1.05 g product was obtained, which was colorless and transparent oil in nature with 44.8% yield. ¹H-NMR (500 MHz, Chloroform-d) δ 7.89 (m, 2H), 7.73-7.65 (m, 4H), 7.45 (m, 2H), 4.31-4.25 (m, 3H), 4.07 (m, 4H), 3.57 (t, J=6.6 Hz, 2H), 2.87 (2H, t, J=6.5 Hz), 2.63 (2H, t, J=6.5 Hz), 2.16 (t, J=7.3 Hz, 2H), 1.54 (s, 1H), 1.53-1.45 (m, 2H), 1.45-1.38 (m, 2H), 1.32-1.15 (m, 4H).

### 1.4 Coupling of Side Chains:

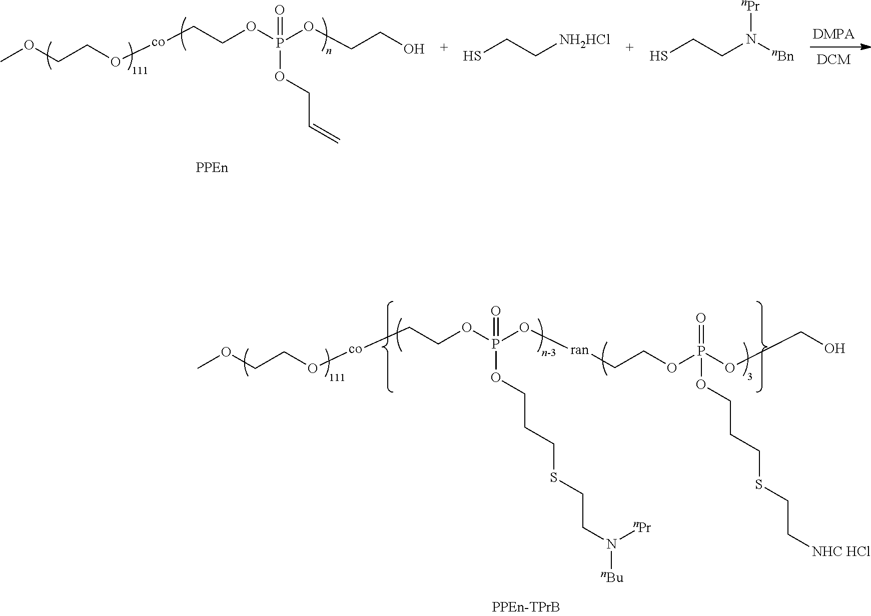

### 1.4.1 PPE70-TPrB (IB003-167-01)

In a glove box with H₂O and O₂ indexes less than 0.1 ppm, PPE70 (255 mg, 0.015 mmol) was dissolved in 4.5 mL dichloromethane, cysteamine hydrochloride (5.12 mg, 0.045 mmol) was added, and then DMPA (25 mg, 10% wt) was added. Under the irradiation of 365 nm ultraviolet lamp, the reaction was carried out under stirring for 1 h at room temperature. TPrB (222.4 mg, 1.05 mmol) was added, and then DMPA (25 mg, 10% wt) was added. Under the irradiation of 365 nm ultraviolet lamp, the reaction was carried out under stirring for 1 h at room temperature, and the reaction system was removed from the glove box. The solvent was removed by rotary evaporation, 10 mL of 50% ethanol was added, and ultrafiltration was performed by using an ultrafiltration centrifuge tube for 45 min and repeated three times. After concentration by rotary evaporation and vacuum drying, a white solid product of 434.6 mg was obtained in 90.6% yield. ¹H NMR (400 MHz, CDCl₃) δ 4.31-4.22 (m, 420H), 3.63 (s, 448H), 3.34-3.31 (m, 143H), 3.14-3.08 (m, 268H), 2.89-2.87 (m, 140H), 2.70-2.68 (m, 140H), 2.00-1.97 (m, 140H), 1.61 (m, 268H), 1.29-1.26 (m, 134H), 0.87-0.81 (m, 402H).

### 1.4.2 PPE90-TPrB

Synthesis and purification of PPE90-TPrB were carried out according to the process as in Example 1.4.1 above, wherein PPE70 was replaced with an equimolar amount of PPE90, and TPrB with a corresponding molar ratio was used, to obtain 205 mg of white solid polymer in 88.4% yield. ¹H NMR (400 MHz, CDCl₃) δ 4.31-4.22 (m, 540H), 3.63 (s, 448H), 3.34-3.30 (m, 183H), 3.15-3.07 (m, 348H), 2.89-2.87 (m, 180H), 2.70-2.67 (m, 180H), 2.00-1.97 (m, 180H), 1.61 (m, 348H), 1.29-1.26 (m, 174H), 0.87-080 (m, 522H).

### 1.4.3 PPE120-TPrB

Synthesis and purification of PPE120-TPrB were carried out according to the procedure of Example 1.4.1 above, in which PPE70 was replaced by PPE120 with equal molar amount, and a proportional molar amount of TPrB was used, to obtain 104 mg of white solid polymer with a yield of 87.8%. ¹H NMR (400 MHz, CDCl₃) δ 4.32-4.27 (m, 738H), 3.63 (s, 448H), 3.33-3.28 (m, 255H), 3.20-3.14 (m, 480H), 2.89-2.86 (m, 246H), 2.75-2.69 (m, 246H), 2.00-1.96 (m, 246H), 1.63-1.60 (m, 480H), 1.31-1.27 (m, 240H), 0.84-0.79 (m, 720H).

### 1.4.4 PPE150-TPrB

Synthesis and purification of PPE150-TPrB were carried out according to the process as in Example 1.4.1 above, wherein PPE70 was replaced with an equimolar amount of PPE150, and a proportional molar amount of TPrB was used, to obtain 205 mg of white solid in 86.3% yield. ¹H NMR (400 MHz, CDCl₃) δ 4.31-4.22 (m, 876H), 3.63 (s, 448H), 3.34-3.30 (m, 295H), 3.14-3.07 (m, 572H), 2.88-2.87 (m, 292H), 2.70-2.67 (m, 292H), 1.99-1.97 (m, 292H), 1.60 (m, 572H), 1.29-1.26 (m, 286H), 0.87-080 (m, 858H).

### 1.4.5 PPE200-TPrB (IB002-086-01)

Synthesis and purification of PPE200-TPrB were carried out according to the process as in Example 1.4.1 above, wherein PPE70 was replaced with an equimolar amount of PPE200, and a proportional molar amount of TPrB was used, to obtain 323 mg of white solid in 84.3% yield. ¹H NMR (400 MHz, D₂O) δ 4.14-3.89 (m, 1244H), 3.37 (s, 448H), 3.15-2.36 (m, 2292H), 1.80-0.97 (m, 1758H), 0.83 (dt, J=13.9, 7.4 Hz, 1273H).

### 1.4.6 PPE250-TPrB

Synthesis and purification of PPE250-TPrB were carried out according to the procedure of Example 1.4.1 above, in which PPE70 was replaced by PPE250 with equal molar amount, and a proportional molar amount of TPrB was used, to obtain 205 mg of white solid polymer with a yield of 86.3%. ¹H NMR (400 MHz, CDCl₃) δ 4.30-4.21 (m, 1548H), 3.63 (s, 448H), 3.34-3.31 (m, 519H), 3.15-3.07 (m, 1020H), 2.88-2.87 (m, 516H), 2.71-2.68 (m, 516H), 2.00-1.96 (m, 516H), 1.61 (m, 1020H), 1.30-1.26 (m, 510H), 0.87-080 (m, 1530H).

### 1.4.7 PPE300-TPrB

Synthesis and purification of PPE300-TPrB were carried out according to the process as in Example 1.4.1 above, wherein PPE70 was replaced with an equimolar amount of PPE300 and TPrB with a corresponding molar ratio was used, to obtain 526 mg of white solid polymer in 92.6% yield. ¹H NMR (400 MHz, CDCl₃) δ 4.32-4.21 (m, 1740H), 3.63 (s, 448H), 3.34-3.30 (m, 583H), 3.15-3.07 (m, 1148H), 2.89-2.87 (m, 580H), 2.70-2.67 (m, 580H), 1.99-1.97 (m, 580H), 1.60 (m, 1148H), 1.29-1.26 (m, 574H), 0.87-080 (m, 1722H).

### 1.4.8 PPE200-TPrB-40C5

The synthetic route for PPE200-TPrB40C5 is shown below, and the synthesis and purification was carried out according to the process as in Example 1.4.1 above, wherein PPE70 was replaced with an equimolar amount of PPE200, and proportional molar amounts of TPr and C₅H₁₁SH were used, to obtain 176 mg of white solid in 72.6% yield. ¹H NMR (400 MHz, D₂O) δ 4.31-4.22 (m, 1200H), 3.68 (s, 448H), 3.35-3.30 (m, 323H), 3.16-3.12 (m, 628H), 2.91-2.88 (m, 320H), 2.70-2.67 (m, 480H), 1.99-1.95 (m, 400H), 1.64 (m, 628H), 1.33-1.28 (m, 554H), 0.86-078 (m, 1062H).

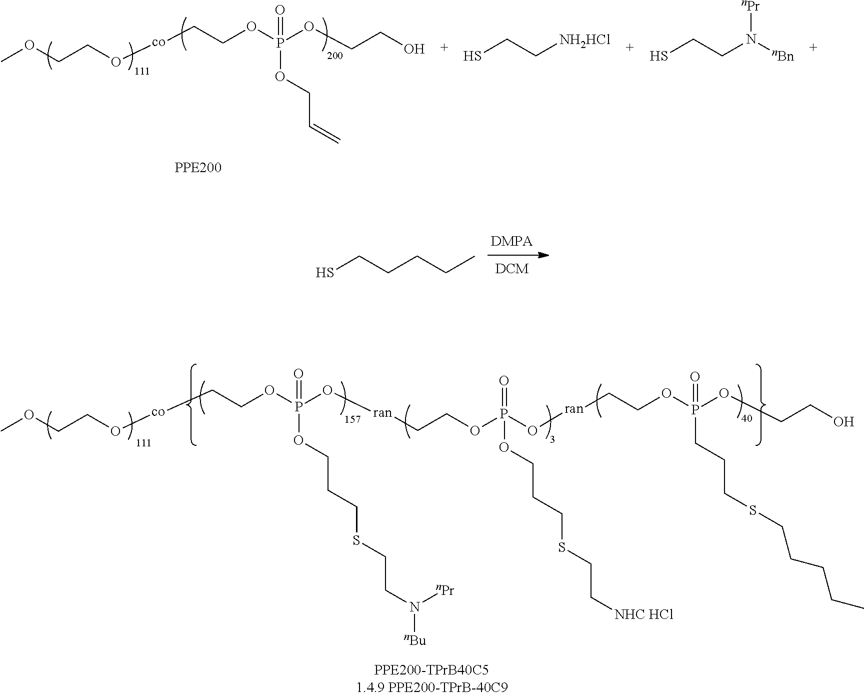

The synthetic route for PPE200-TPrB40C9 is shown below, and the synthesis and purification was carried out according to the process as in Example 1.4.1 above, wherein PPE70 was replaced with an equimolar amount of PPE200, and proportional molar amounts of TPr and C9H19SH were used, to obtain 191 mg of white solid in 80.2% yield. ¹H NMR (400 MHz, D₂O) δ 4.32-4.20 (m, 1200H), 3.68 (s, 448H), 3.35-3.30 (m, 320H), 3.15-3.07 (m, 628H), 2.89-2.87 (m, 320H), 2.72-2.68 (m, 480H), 2.00-1.96 (m, 400H), 1.60 (m, 628H), 1.29-1.27 (m, 874H), 0.85-0.77 (m, 1062H).

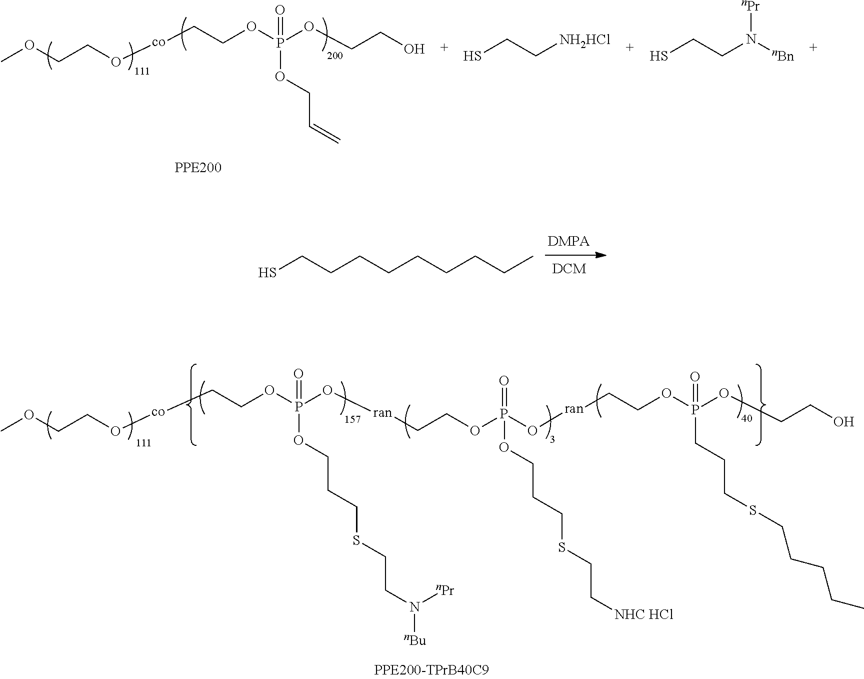

### 1.4.10 PPE200-TPrB-80C9

The synthetic route for PPE200-TPrB80C9 is shown below, and the synthesis and purification was carried out according to the process as in Example 1.4.1 above, wherein PPE70 was replaced with an equimolar amount of PPE200, and proportional molar amounts of TPr and C9H19SH were used, to obtain 202 mg of white solid in 83.8% yield. ¹H NMR (400 MHz, D₂O) δ 4.32-4.20 (m, 1200H), 3.66 (s, 448H), 3.34-3.29 (m,. 243H), 3.13-3.08 (m, 468H), 2.90-2.84 (m, 240H), 2.69 (m, 560H), 2.00-1.95 (m, 400H), 1.61 (m, 468H), 1.29-1.26(m, 1354H), 0.86-0.78 (m, 942H).

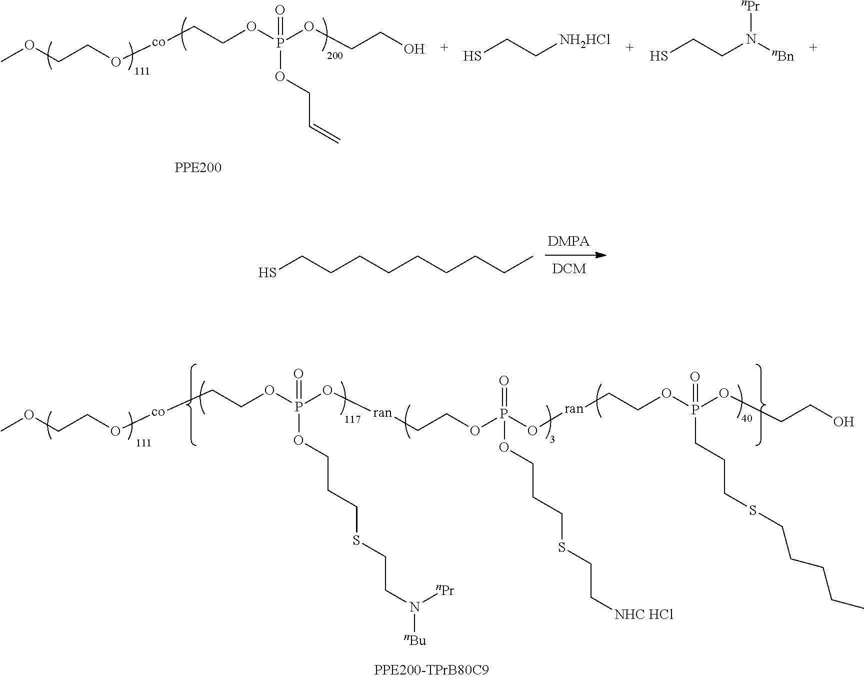

### 1.4.11 PPE90-TEPr

Synthesis and purification of PPE90-TEPr were carried out according to the process as in Example 1.4.2 above (TPrB was replaced with an equimolar amount of TEPr, the specific chemical reaction is shown below) to give 130 mg of white solid in 60.5% yield. ¹H NMR (400 MHz, CDCl₃) δ 4.33-4.22 (m, 540H), 3.61 (s, 448H), 3.34-3.27 (m, 183H), 3.18-3.09 (m, 348H), 2.90-2.87 (m, 180H), 2.76-2.66 (m, 180H), 2.00-1.97 (m, 180H), 1.62 (m, 174H), 1.26-1.23 (m, 126H), 0.88-0.81 (m, 261H).

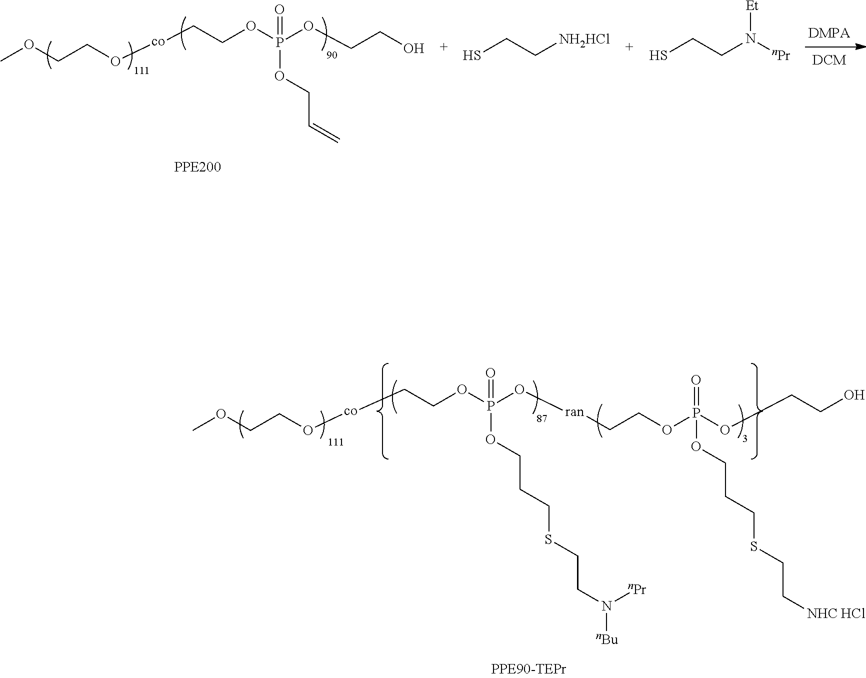

### 1.4.12 PPE90-TPrPr

Synthesis and purification of PPE90-TPrPr were carried out according to the process as in Example 1.4.2 above (TPrB was replaced with an equimolar amount of TPrPr, the specific chemical reaction is shown below) to give 119 mg of white solid in 65.5% yield. ¹H NMR (400 MHz, D₂O) δ 4.32-4.25 (m, 540H), 3.65 (s, 448H), 3.37-3.31 (m, 183H), 3.11-3.08 (m, 348H), 2.90-2.88 (m, 180H), 2.72-2.67 (m, 180H), 2.02-1.99 (m, 180H), 1.64-1.58 (m, 348H), 0.84-0.81, (m, 522H).

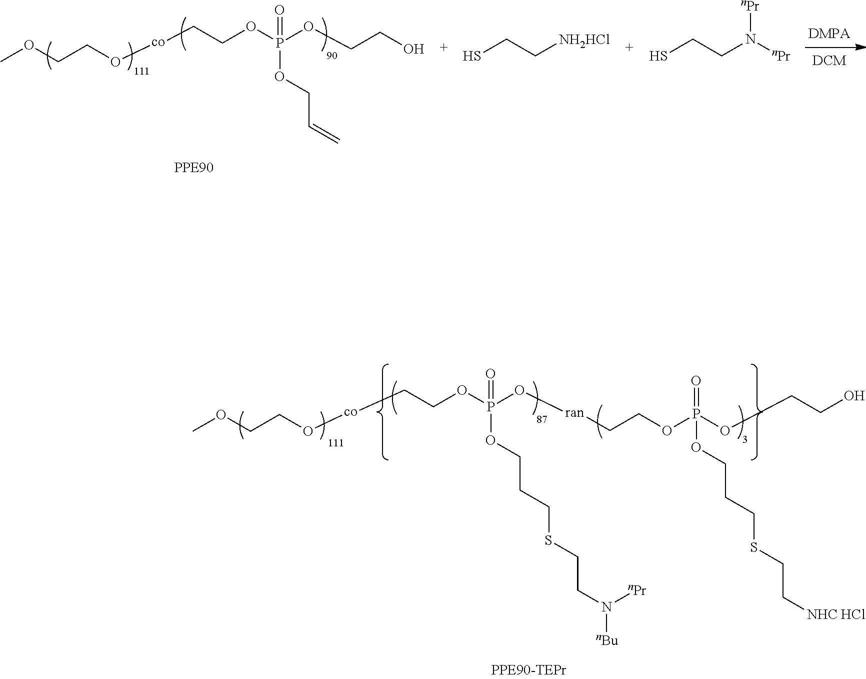

### 1.4.13 PPE90-TBB

Synthesis and purification of PPE90-TBB were carried out according to the process as in Example 1.4.2 above (TPrB was replaced with an equimolar amount of TBB, the specific chemical reaction is shown below) to give 150 mg of white solid in 63.6% yield. ¹H NMR (400 MHz, CDCl₃) δ 4.33-4.23 (m, 540H), 3.66 (s, 448H), 3.34-3.31 (m, 183H), 3.15-3.08 (m, 348H), 2.90-2.88 (m, 180H), 2.73-2.68 (m, 180H), 2.02-1.94 (m, 180H), 1.59 (m, 348H), 1.26-1.23 (m, 348H), 0.84-0.81 (m, 522H).

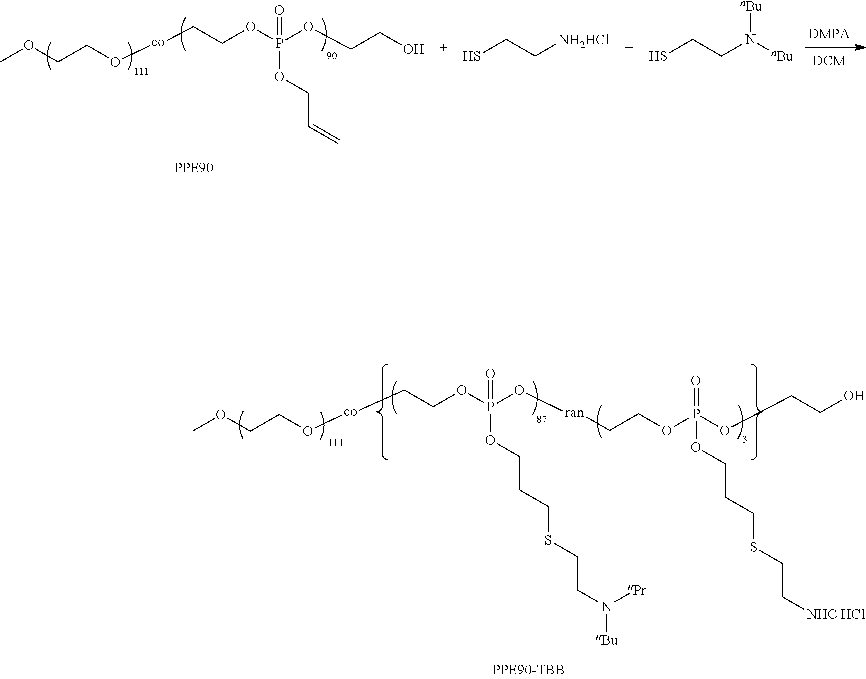

### 1.4.14 PPE90-TBPe

Synthesis and purification of PPE90-TBPe were carried out according to the process as in Example 1.4.2 above (TPrB was replaced with an equimolar amount of TBPe, the specific chemical reaction is shown below) to give 80 mg of white solid in 63.5% yield. ¹H NMR (400 MHz, CDCl₃) δ 4.33-4.23 (m, 540H), 3.66 (s, 448H), 3.34-3.31 (m, 183H), 3.15-3.08 (m, 348H), 2.90-2.88 (m, 180H), 2.73-2.68 (m, 180H), 2.02-1.94 (m, 180H), 1.59 (m, 348H), 1.26-1.23 (m, 348H), 0.84-0.81 (m, 522H).

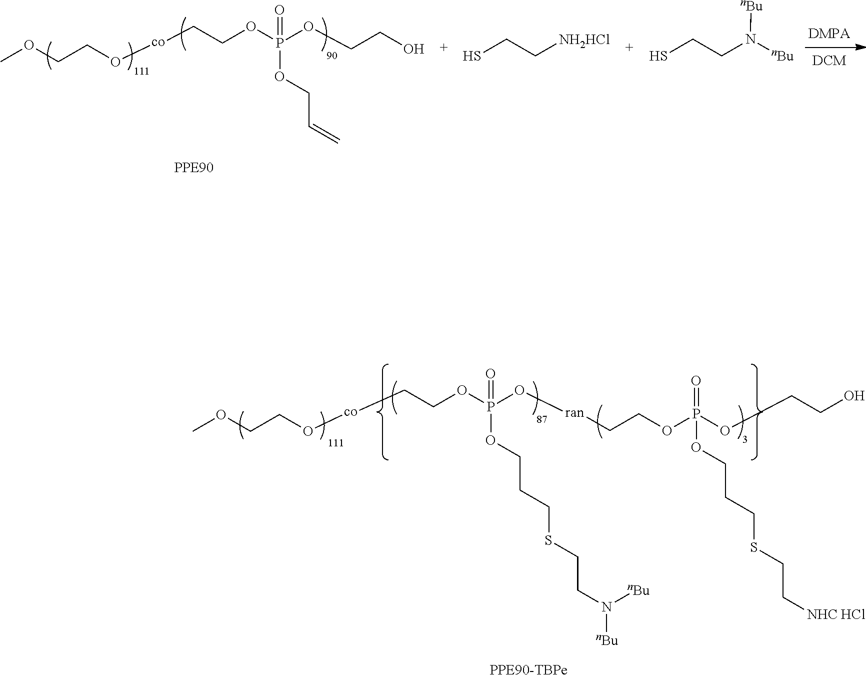

### 1.4.15 PPE90-TPePe

Synthesis and purification of PPE90-TPePe were carried out according to the process as in Example 1.4.2 above (TPrB was replaced with an equimolar amount of TPePe, the specific chemical reaction is shown below) to give 165 mg of white solid in 73.9% yield. ¹H NMR (400 MHz, CDCl₃) δ 4.31-4.21 (m, 540H), 3.63 (s, 448H), 3.34-3.31 (m, 183H), 3.14-3.07 (m, 348H), 2.89-2.87 (m, 180H), 2.70-2.67 (m, 180H), 2.00-1.96 (m, 180H), 1.61 (m, 348H), 1.30-1.26 (m, 696H), 0.87-0.80 (m, 522H).

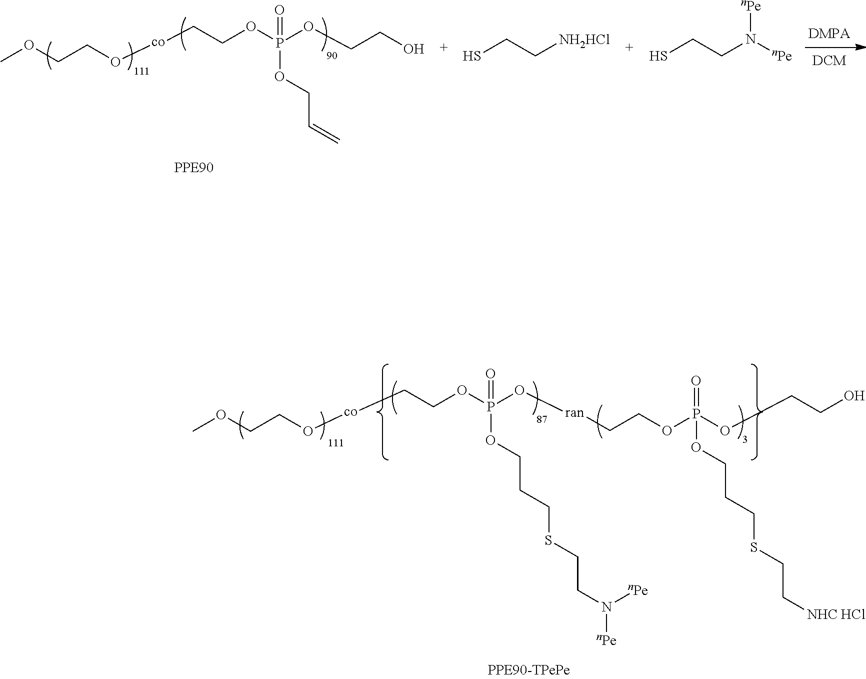

### 1.4.16 PPE90-THH

Synthesis and purification of PPE90-THH were carried out according to the process as in Example 1.4.2 above (TPrB was replaced with an equimolar amount of THH, the specific chemical reaction is shown below) to give 176 mg of white solid in 72.6% yield. ¹H NMR (400 MHz, CDCl₃) δ 4.32-4.22 (m, 540H), 3.63 (s, 448H), 3.34-3.30 (m, 183H), 3.15-3.07 (m, 348H), 2.88-2.87 (m, 180H), 2.71-2.67 (m, 180H), 2.00-1.97 (m, 180H), 1.60 (m, 348H), 1.29-1.26 (m, 1044H), 0.87-080 (m, 522H).

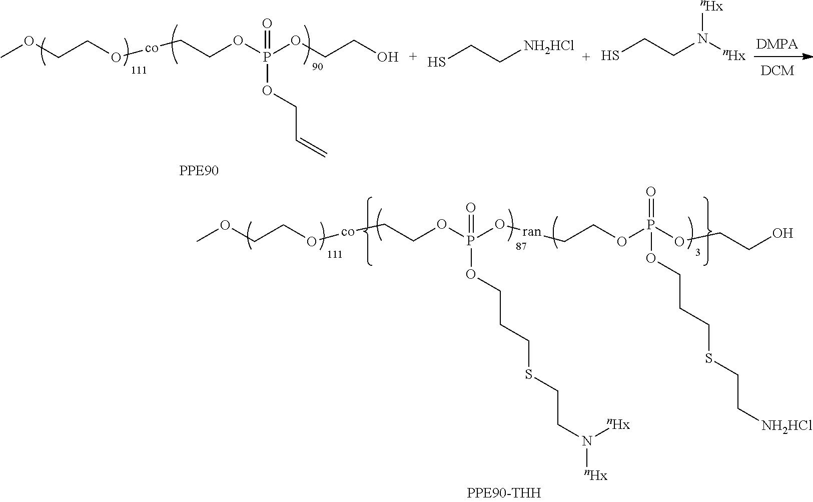

### 1.4.17 OH-PPE90-TPrB

Synthesis and purification of OH-PPE90-TPrB were carried out according to the process as in Example 1.4.2 above (PPE90 was replaced with an equimolar amount of OH-PPE90 and an equimolar amount of TPrB was used, the specific chemical reaction is shown below) to give 176 mg of white solid in 72.6% yield. ¹H NMR (400 MHz, CDCl₃) δ 4.31-4.22 (m, 540H), 3.62 (s, 448H), 3.34-3.31 (m, 180H), 3.12 (m, 348H), 2.88 (m, 180H), 2.70-2.66 (m, 180H), 2.01-1.98 (m, 180H), 1.61 (m, 348H), 1.29-1.26 (m, 174H) 0.87-0.79 (m, 522H).

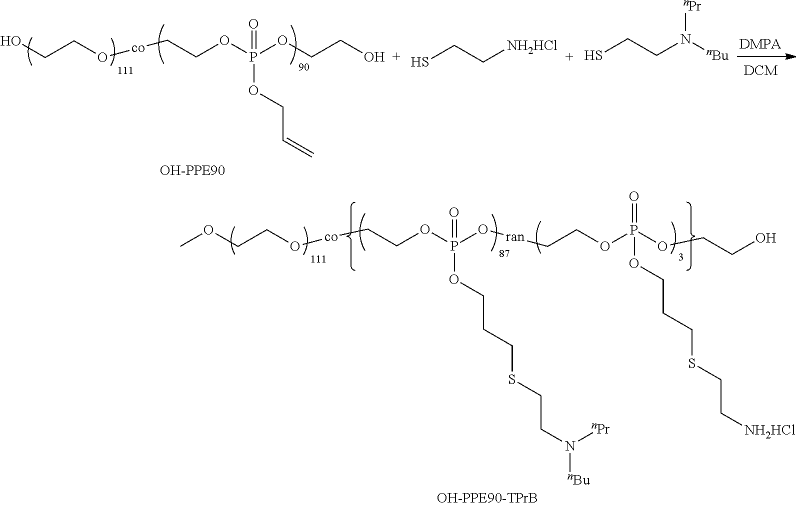

### 1.4.18 PPE90-TPrB-FmocALA10

Synthesis and purification of PPE90-TEPr-FmocALA10 were carried out according to the process as in Example 1.4.2 above (TPrB was replaced with a 77 molar amount of TEPr and a 10 molar amount of 6-mercaptohexyl 5-Fmoc-5-amino-4-oxopentanoate, with the specific chemistry as shown below) to give 185 mg of white solid with a yield of 71.9%. ¹H NMR (400 MHz, CDCl₃) δ 7.87 (m, 20H), 7.73-7.63 (m, 40H), 7.44 (m, 20H), 4.31-4.22 (m, 570H), 4.01 (br, 40H), 3.82-3.55 (m, 488H), 3.34-3.31 (m, 157H), 3.12 (m, 308H), 2.87 (m, 200H), 2.70-2.61 (m, 200H), 2.12 (t, J=7.2 Hz, 20H), 2.01-1.98 (m, 180H), 1.61-1.41 (m, 338H), 1.29-1.14 (m, 254H), 0.87-0.79 (m, 522H).

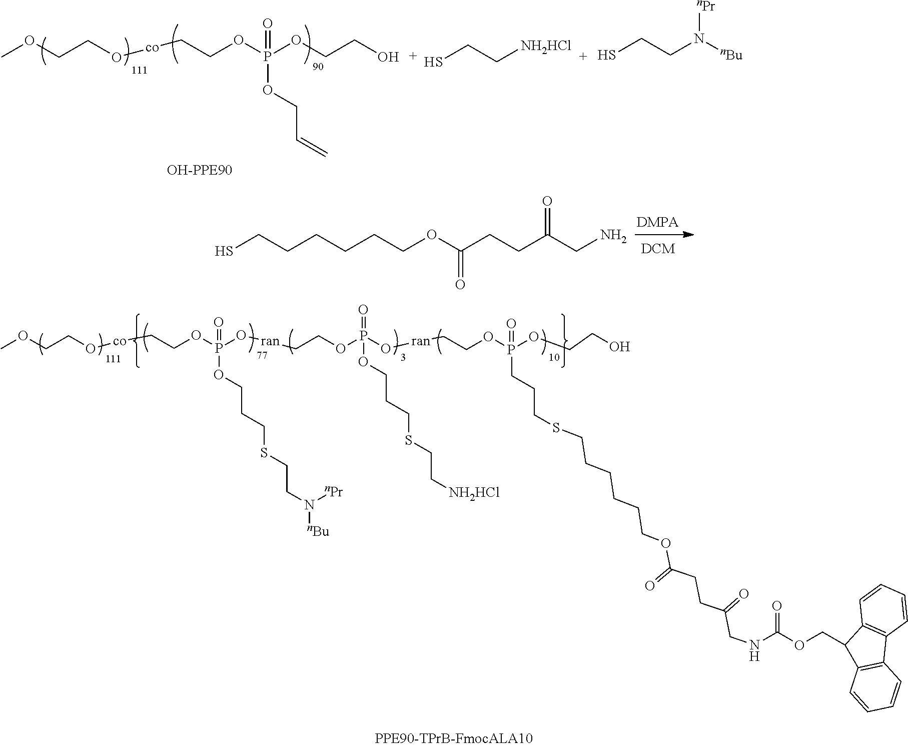

### 1.5 Coupling of Fluorescent Molecules:

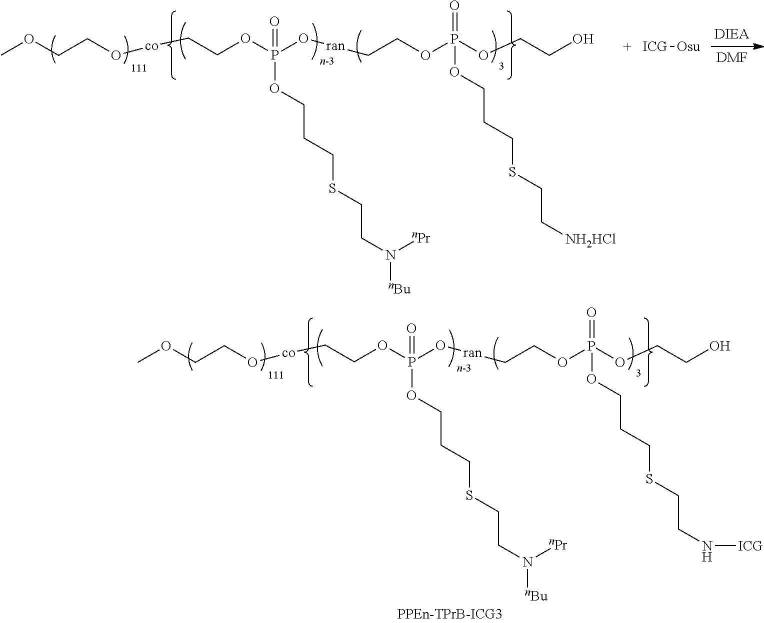

### 1.5.1 PPE70-TPrB-ICG3

Polymer PPE70-TPrB (125 mg, 0.0069 mmol) was dissolved in 2 ml DMF, ICG-Osu (25.8 mg, 0.031 mmol) and DIEA (51 mg, 0.396 mmol) were sequentially added, and then the mixture was stirred overnight at room temperature. DMF was removed by concentration, the residue was dissolved in 100 ml absolute ethanol, purified by ceramic membrane (5K) for 2 hours, and EtOH was removed by concentration. After vacuum drying, 92 mg of polymer was obtained, which was a dark green solid with a yield of 73.1%. ¹H NMR (400 MHz, CDCl₃) δ 8.12-7.47 (m, 45H), 6.74-6.41 (m, 12H), 4.36-4.23 (m, 420H), 3.64 (s, 448H), 3.38-3.30 (m, 140H), 3.12-3.07 (m, 268H), 2.93-2.88 (m, 140H), 2.72-2.66 (m, 140H), 1.99-1.31 (m, 632H), 0.89-0.80 (m, 402H).

### 1.5.2 PPE90-TPrB-ICG3

Synthesis and purification of PPE90-TPrB-ICG3 were carried out according to the process as in Example 1.5.1 above (PPE70-TPrB was replaced with an equimolar amount of PPE90-TPrB) to give 77.2 mg of polymer as a dark green solid in 80.9% yield. ¹H NMR (400 MHz, CDCl₃) δ 8.12-7.47 (m, 45H), 6.74-6.44 (m, 12H), 4.31-4.22 (m, 540H), 3.63 (s, 448H), 3.34-3.31 (m, 186H), 3.14-3.08 (m, 348H), 2.89-2.87 (m, 180H), 2.70-2.68 (m, 180H), 2.02-1.28 (m, 792H), 0.87-080 (m, 522H).

### 1.5.3 PPE120-TPrB-ICG3

Synthesis and purification of PPE120-TPrB-ICG3 were carried out according to the process as in Example 1.5.1 above (PPE70-TPrB was replaced with an equimolar amount of PPE120-TPrB) to give 45.3 mg of polymer as a dark green solid in 81.4% yield. ¹H NMR (400 MHz, CDCl₃) δ 8.09-7.45 (m, 45H), 6.75-6.47 (m, 12H), 4.35-4.20 (m, 738H), 3.64 (s, 448H), 3.37-3.35 (m, 252H), 3.20-3.04 (m, 480H), 2.92-2.89 (m, 246H), 2.68-2.65 (m, 246H), 2.02-1.26 (m, 1056H), 0.88-0.79 (m, 720H).

### 1.5.4 PPE150-TPrB-ICG3

Synthesis and purification of PPE150-TPrB-ICG3 were carried out according to the process as in Example 1.5.1 above (PPE70-TPrB was replaced with an equimolar amount of PPE150-TPrB) to give 51 mg of polymer as a dark green solid in 79.8% yield. ¹H NMR (400 MHz, CDCl₃) δ 8.10-7.47 (m, 45H), 6.77-6.49 (m, 12H), 4.34-4.20 (m, 876H), 3.61 (s, 448H), 3.35-3.32 (m, 298H), 3.17-3.06 (m, 572H), 2.90-2.86 (m, 292H), 2.72-2.68 (m, 292H), 2.00-1.26 (m, 1240H), 0.85-0.79 (m, 858H).

### 1.5.5 PPE200-TPrB-ICG3 (IB002-091-01)

Synthesis and purification of PPE200-TPrB-20C5-ICG3 were carried out according to the process as in Example 1.5.1 above (PPE70-TPrB was replaced with an equimolar amount of PPE200-TPrB) to give 60 mg of polymer as a dark green solid in 72.6% yield. ¹H NMR (400 MHz, CDCl₃) δ 8.09-7.48 (m, 45H), 6.76-6.49 (m, 12H), 4.33-4.25 (m, 1218H), 3.65 (s, 448H), 3.38-3.35 (m, 415H), 3.17-3.04 (m, 800H), 2.92-2.89 (m, 406H), 2.72-2.69 (m, 406H), 2.00-1.27 (m, 1696H), 0.89-0.80 (m, 1200H).

### 1.5.6 PPE250-TPrB-ICG3

Synthesis and purification of PPE250-TPrB-ICG3 were carried out according to the process as in Example 1.5.1 above (PPE70-TPrB was replaced with an equimolar amount of PPE250-TPrB) to give 59.3 mg of polymer as a dark green solid in 79.6% yield. ¹H NMR (400 MHz, CDCl₃) δ 8.10-7.47 (m, 45H), 6.73-6.46 (m, 12H), 4.32-4.23 (m, 1548H), 3.63 (s, 448H), 3.33-3.29 (m, 5252H), 3.19-3.08 (m, 1020H), 2.91-2.87 (m, 516H), 2.70-2.65 (m, 516H), 2.02-1.26 (m, 2136H), 0.87-0.79 (m, 1530H).

### 1.5.7 PPE300-TPrB-ICG3

Synthesis and purification of PPE300-TPrB-ICG3 were carried out according to the process as in Example 1.5.1 above (PPE70-TPrB was replaced with an equimolar amount of PPE300-TPrB) to give 92 mg of polymer as a dark green solid in 86.7% yield. ¹H NMR (400 MHz, CDCl₃) δ 8.12-7.51 (m, 45H), 6.734-6.42 (m, 12H), 4.36-4.22 (m, 1740H), 3.65 (s, 448H), 3.37-3.34 (m, 589H), 3.16-3.11 (m, 1148H), 2.91-2.89 (m, 580H), 2.71-2.69 (m, 580H), 2.02-1.24 (m, 2392H), 0.87-0.83 (m, 1722H).

### 1.5.8 PPE200-TPrB-40C5-ICG3

Synthesis and purification of PPE200-TPrB-40C5-ICG3 were carried out according to the process as in Example 1.5.1 above (PPE70-TPrB was replaced with an equimolar amount of PPE200-TPrB-40C5, with the specific chemistry as shown below) to give 60 mg of polymer as a dark green solid in 72.6% yield. ¹H NMR (400 MHz, CDCl₃) δ 8.16-7.47 (m, 45H), 6.74-6.49 (m, 12H), 4.32-4.25 (m, 1200H), 3.63 (s, 448H), 3.35-3.31 (m, 363H), 3.18-3.09 (m, 708H), 2.90-2.87 (m, 360H), 2.72-2.66 (m, 440H), 2.00-1.30 (m, 1672H), 0.88-0.78 (m, 1122H).

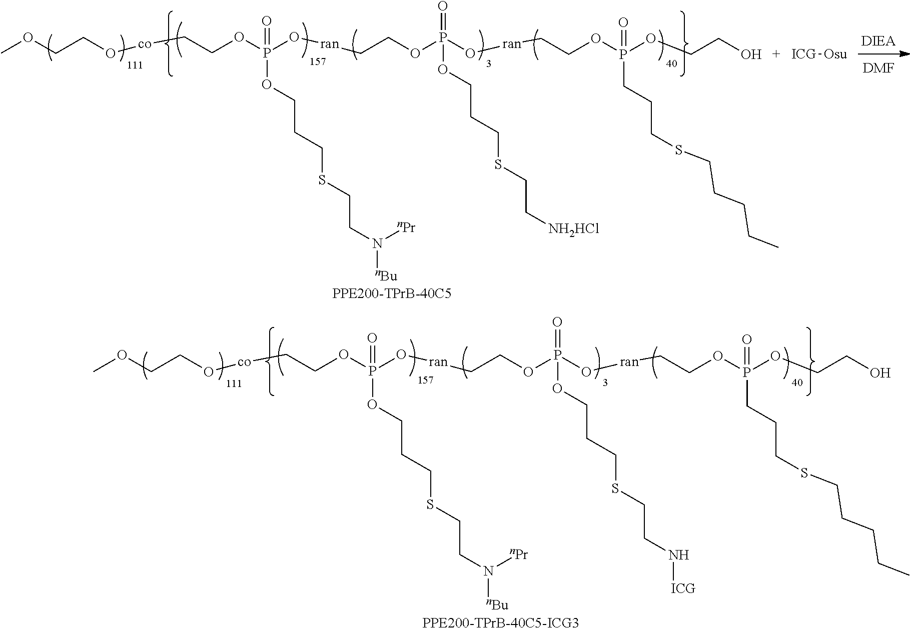

### 1.5.9 PPE200-TPrB-40C9-ICG3

Synthesis and purification of PPE200-TPrB-40C9-ICG3 were carried out according to the process as in Example 1.5.1 above (PPE70-TPrB was replaced with an equimolar amount of PPE200-TPrB-40C9, with the specific chemistry as shown below) to give 52.1 mg of polymer as a dark green solid in 88.1% yield. ¹H NMR (400 MHz, CDCl₃) δ 8.10-7.46 (m, 45H), 6.78-6.47 (m, 12H), 4.35-4.26 (m, 1200H), 3.63 (s, 448H), 3.39-3.30 (m, 323H), 3.20-3.12 (m, 628H), 2.91-2.86 (m, 320H), 2.72-2.66 (m, 480H), 2.04-1.28 (m, 1992H), 0.87-0.82 (m, 1062H).

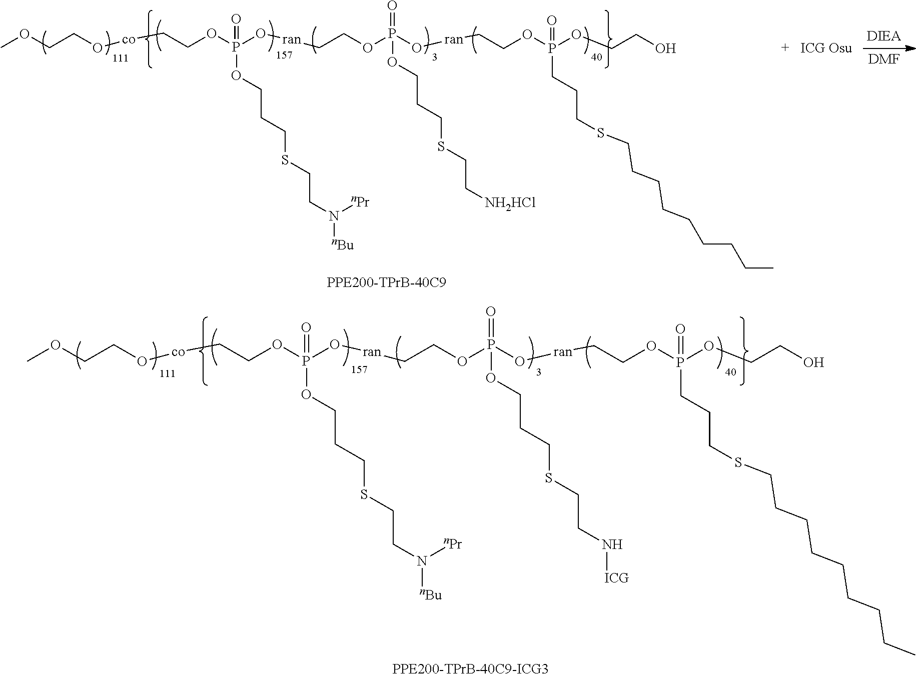

### 1.5.10 PPE200-TPrB-80C9-ICG3

Synthesis and purification of PPE200-TPrB-80C9-ICG3 were carried out according to the process as in Example 1.5.1 above (PPE70-TPrB was replaced with an equimolar amount of PPE200-TPrB-80C9, with the specific chemistry as shown below) to give 77 mg of polymer as a dark green solid in 74.2% yield. ¹H NMR (400 MHz, CDCl₃) δ 8.13-7.50 (m, 45H), 6.72-6.48 (m, 12H), 4.36-4.27 (m, 1200H), 3.63 (s, 448H), 3.36-3.33 (m, 243H), 3.14-3.04 (m, 468H), 2.90-2.88 (m, 240H), 2.72-2.69 (m, 560H), 2.04-1.29 (m, 2312H), 0.89-0.81 (m, 942H).

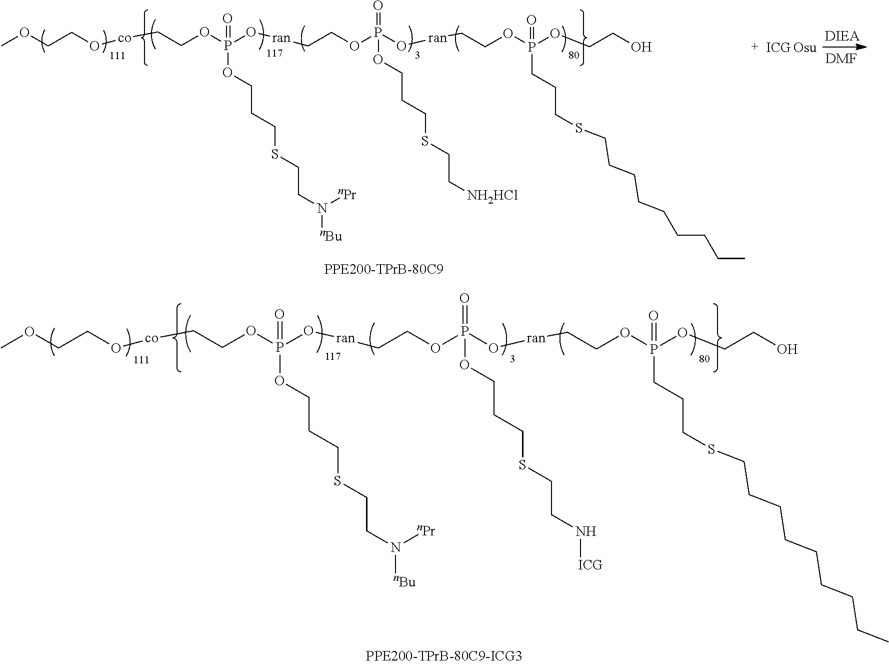

### 1.5.11 PPE90-TEPr-ICG3

Synthesis and purification of PPE90-TEPr-ICG3 were carried out according to the process as in Example 1.5.1 above (PPE70-TPrB was replaced with an equimolar amount of PPE90-TEPr, with the specific chemistry as shown below) to give 90 mg of polymer as a dark green solid in 95.4% yield. ¹H NMR (400 MHz, CDCl₃) δ 8.11-7.51 (m, 45H), 6.72-6.47 (m, 12H), 4.29-4.20 (m, 540H), 3.61 (s, 448H), 3.33-3.30 (m, 183H), 3.15-3.10 (m, 348H), 2.89-2.87 (m, 180H), 2.70-2.65 (m, 180H), 2.02-1.25 (m, 705H), 0.84-0.79 (m, 261H).

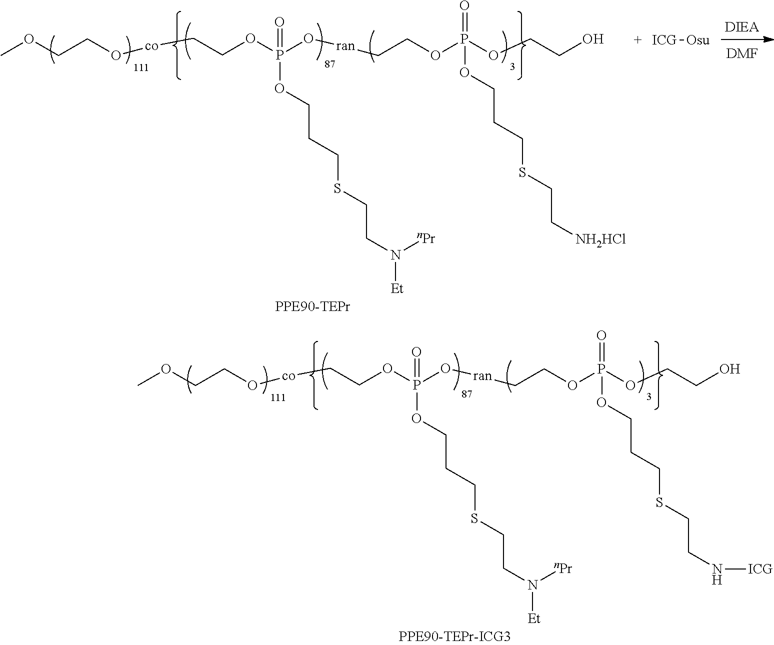

### 1.5.12 PPE90-TPrPr-ICG3

Synthesis and purification of PPE90-TPrPr-ICG3 were carried out according to the process as in Example 1.5.1 above (PPE70-TPrB was replaced with an equimolar amount of PPE90-TPrPr, with the specific chemistry as shown below) to give 90 mg of polymer as a dark green solid in 56.9% yield. ¹H NMR (400 MHz, CDCl₃) δ 8.16-7.44 (m, 45H), 6.73-6.46 (m, 12H), 4.31-4.25 (m, 540H), 3.63 (s, 448H), 3.36-3.32 (m, 183H), 3.17-3.08 (m, 348H), 2.92-2.90 (m, 180H), 2.71-2.66 (m, 180H), 2.00-1.27 (m, 618H), 0.86-0.79 (m, 522H).

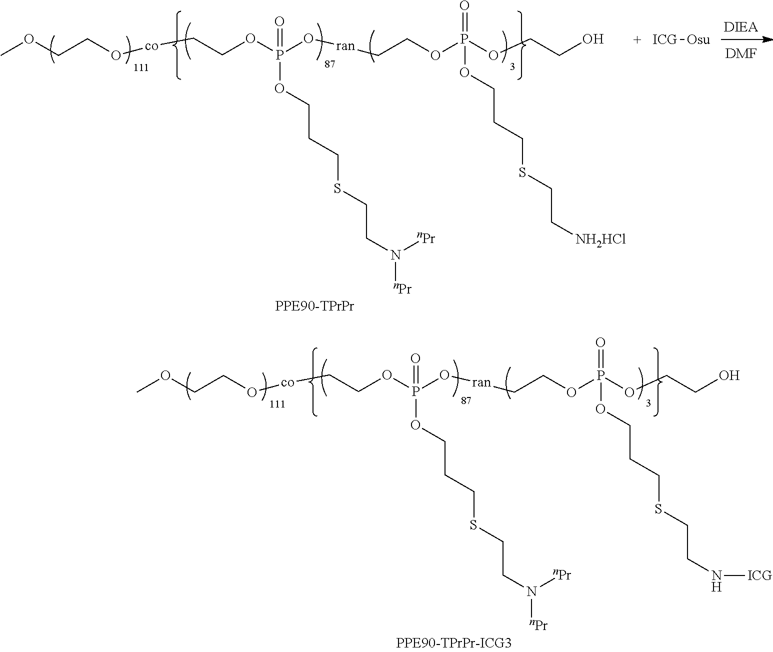

### 1.5.13 PPE90-TBB-ICG3

Synthesis and purification of PPE90-TBB-ICG3 were carried out according to the process as in Example 1.5.1 above (PPE70-TPrB was replaced with an equimolar amount of PPE90-TBB, with the specific chemistry as shown below) to give 38.2 mg of polymer as a dark green solid in 82.5% yield. ¹H NMR (400 MHz, CDCl₃) δ 8.14-7.51 (m, 45H), 6.78-6.44 (m, 12H), 4.33-4.19 (m, 540H), 3.61 (s, 448H), 3.39-3.36 (m, 183H), 3.17-3.09 (m, 348H), 2.92-2.89 (m, 180H), 2.70-2.67 (m, 180H), 2.02-1.31 (m, 996H), 0.90-0.82 (m, 522H).

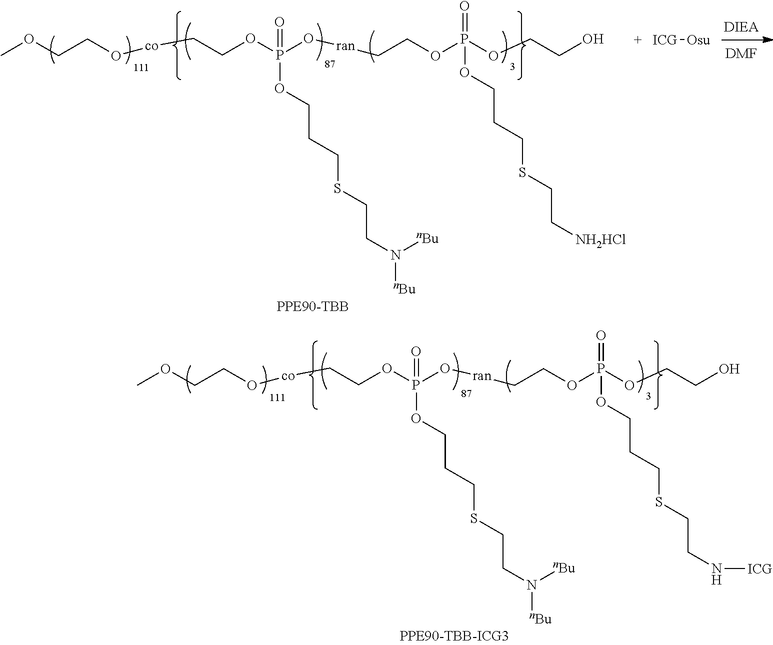

### 1.5.14 PPE90-TBPe-ICG3

Synthesis and purification of PPE90-TBPe-ICG3 were carried out according to the process as in Example 1.5.1 above (PPE70-TPrB was replaced with an equimolar amount of PPE90-TBPe, with the specific chemistry as shown below) to give 33.7 mg of polymer as a dark green solid in 84.7% yield. ¹H NMR (400 MHz, CDCl₃) δ 8.16-7.52 (m, 45H), 6.72-6.46 (m, 12H), 4.34-4.24 (m, 540H), 3.66 (s, 448H), 3.31-3.27 (m, 183H), 3.13-3.10 (m, 348H), 2.92-2.87 (m, 180H), 2.70-2.66 (m, 180H), 2.00-1.31 (m, 1140H), 0.90-0.77 (m, 522H).

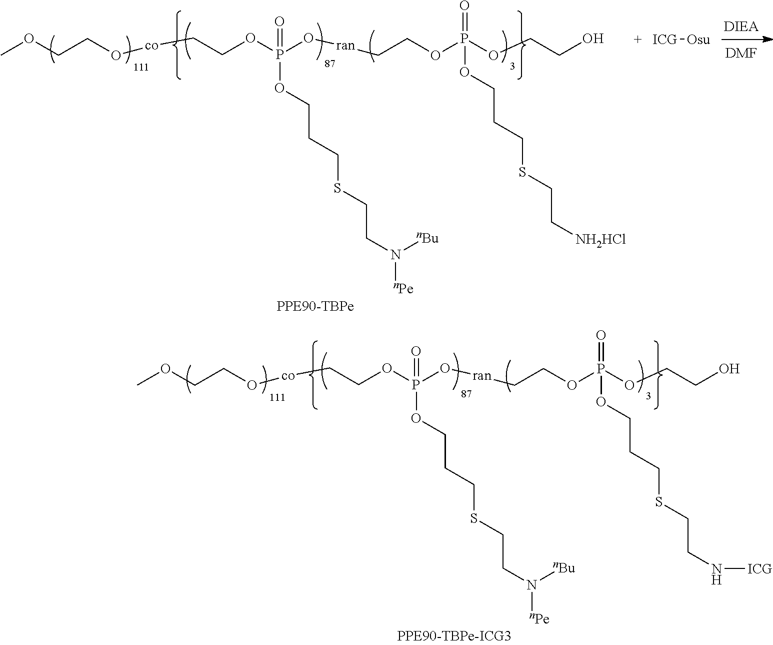

### 1.5.15 PPE90-TPePe-ICG3

Synthesis and purification of PPE90-TPePe-ICG3 were carried out according to the process as in Example 1.5.1 above (PPE70-TPrB was replaced with an equimolar amount of PPE90-TPePe, with the specific chemistry as shown below) to give 35.6 mg of polymer as a dark green solid in 73.2% yield. ¹H NMR (400 MHz, CDCl₃) δ 8.10-7.52 (m, 45H), 6.73-6.45 (m, 12H), 4.33-4.29 (m, 540H), 3.64 (s, 448H), 3.35-3.27 (m, 183H), 3.15-3.10 (m, 348H), 2.89-2.84 (m, 180H), 2.71-2.68 (m, 180H), 2.02-1.28 (m, 1314H), 0.88-0.80 (m, 522H).

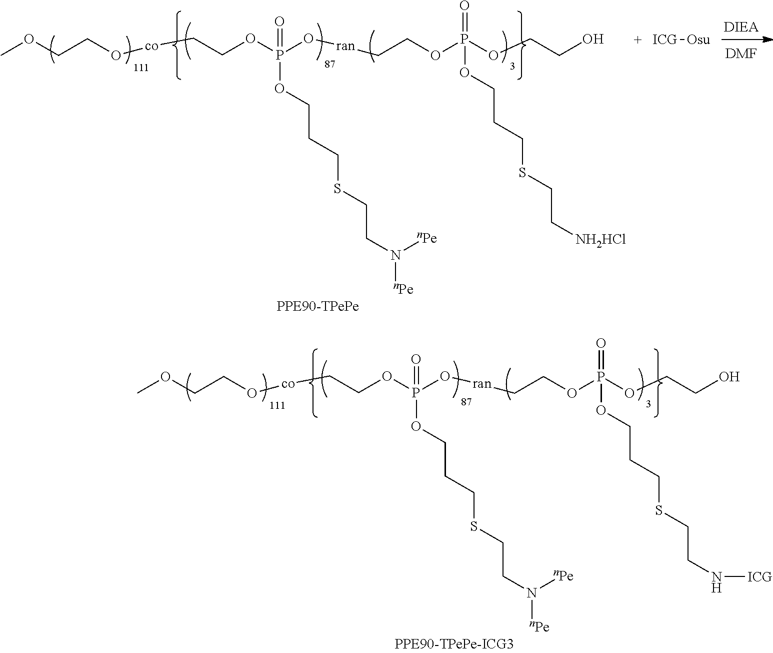

### 1.5.16 PPE90-THH-ICG3

Synthesis and purification of PPE90-THH-ICG3 were carried out according to the process as in Example 1.5.1 above (PPE70-TPrB was replaced with an equimolar amount of PPE90-THH, with the specific chemistry as shown below) to give 36.8 mg of polymer as a dark green solid in 75.4% yield. ¹H NMR (400 MHz, CDCl₃) δ 8.16-7.48 (m, 45H), 6.73-6.45 (m, 12H), 4.30-4.22 (m, 540H), 3.63 (s, 448H), 3.34-3.30 (m, 183H), 3.14-3.09 (m, 348H), 2.89-2.86 (m, 180H), 2.73-2.71 (m, 180H), 2.04-1.27 (m, 1662H), 0.89-0.80 (m, 522H).

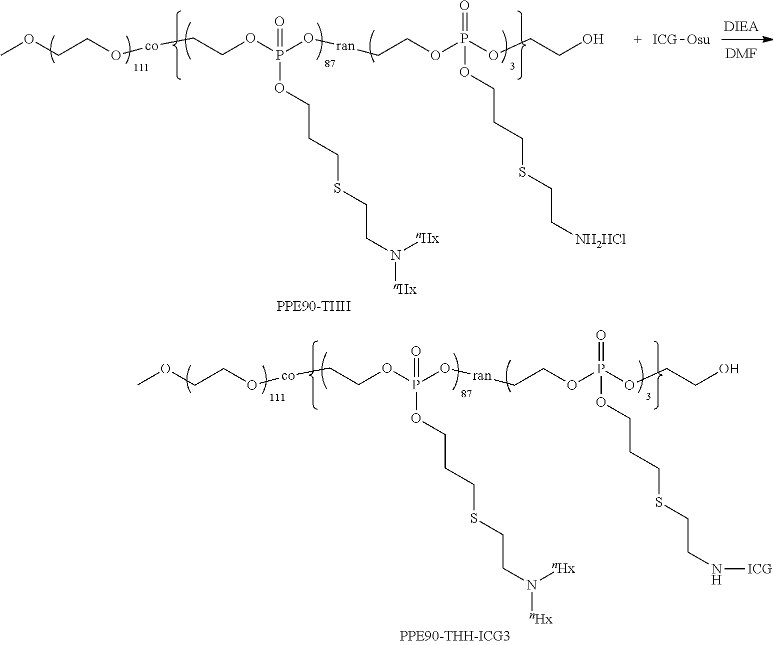

### 1.5.17 OH-PPE90-TPrB-ICG3

Synthesis and purification of OH-PPE90-TPrB-ICG3 were carried out according to the process as in Example 1.5.1 above (PPE70-TPrB was replaced with an equimolar amount of OH-PPE90-TPrB, with the specific chemistry as shown below) to give 37.1 mg of polymer as a dark green solid in 76.5% yield. ¹H NMR (400 MHz, CDCl₃) δ 8.17-7.53 (m, 45H), 6.72-6.45 (m, 12H), 4.33-4.23 (m, 540H), 3.66 (s, 448H), 3.31-3.28 (m, 180H), 3.14-3.10 (m, 348H), 2.94-2.88 (m, 180H), 2.68 (m, 180H), 2.01-1.30 (m, 792H), 0.91-0.78 (m, 522H).

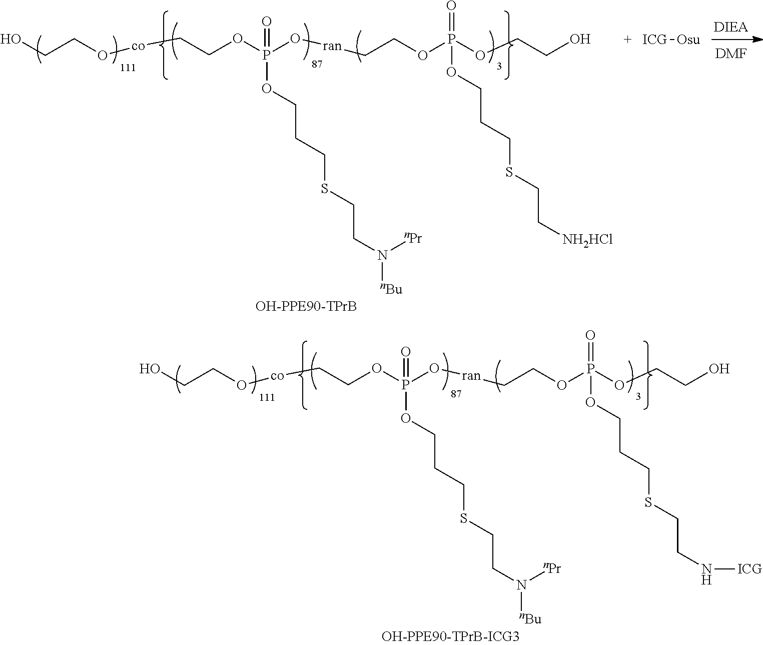

### 1.5.18 PPE90-TPrB-ALA10-ICG3

The synthesis of PPE90-TEPr-ALA10-ICG3 is shown in the following figure. The polymer PPE90-TPrB-Fmoc-ALA10 (150 mg, 0.0039 mmol) is dissolved in 2 ml DMF, and ICG-Osu (5.8 mg, 0.0069 mmol) and DIEA (25 mg, 0.2 mmol) were sequentially added. After stirring overnight at room temperature, DIEA was removed by rotary evaporation, 0.2 ml of piperidine was added, the system was stirred at room temperature for 0.5 hour, DMF was removed by concentration, the residue was dissolved in 100 ml of absolute ethanol, purified by ceramic membrane (5K) for 2 hours, and EtOH was removed by concentration. After vacuum drying, 112 mg of polymer was obtained, which was a dark green solid with a yield of 74.4%. ¹H NMR (400 MHz, CDCl₃) δ 8.17-7.53 (m, 45H), 6.72-6.45 (m, 12H), 4.32-4.20 (m, 570H), 4.04 (br, 20H), 3.82-3.55 (m, 488H), 3.34-3.31 (m, 154H), 3.13 (m, 308H), 2.87 (m, 2000H), 2.70-2.60 (m, 200H), 2.12 (t, J=7.2 Hz, 20H), 2.01-1.98 (m, 180H), 1.61-1.41 (m, 338H), 1.29-1.14 (m, 254H), 0.87-0.79 (m, 522H).

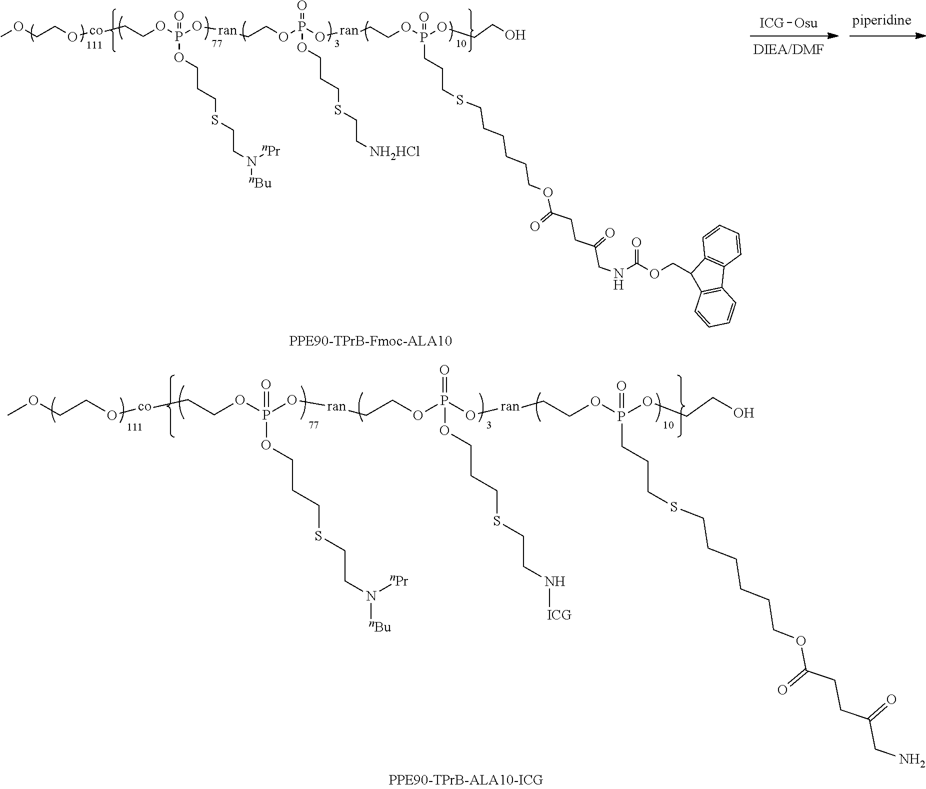

### EXAMPLE 2

### pKa Test

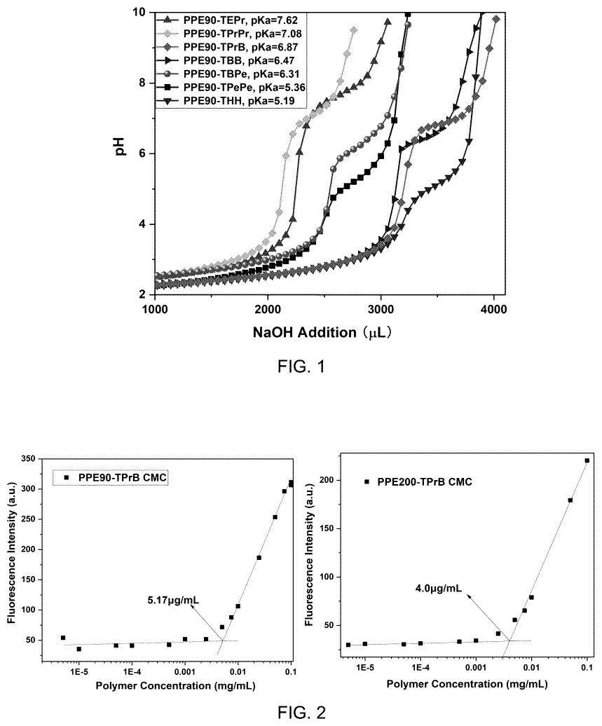

30 mg of the polymer prepared in Example 1 (1.4.3, 1.4.11-1.4.15) was accurately weighed and dissolved in 30 mL of 0.01 mol/L trifluoroacetic acid solution, titrated with 0.1 mol/L sodium hydroxide solution under the indication of a pH meter, and the volume of the consumed sodium hydroxide solution and the corresponding pH were recorded and plotted in terms of the volume versus the pH by Origin software for graphing, and the pKa value is one-half of the sum of the two intersection points of the two tangent lines and the platform tangent. The specific results are shown in FIG. 1. As can be seen from FIG. 1, the pKa of PPE with hydrophilic tertiary amine is greater than that with hydrophobic tertiary amine.

### EXAMPLE 3

### CMC Test of Typical PPE Nano-Fluorescent Probes

2 μL of 1×10⁻⁵ mol/L Nile Red in dichloromethane solution was added to PBS 8.0 solutions of the polymer (Examples 1.4.1 & 1.4.5) at a series of concentrations (1×10⁻⁶˜1×10⁻¹ mg/mL), and the mixture was mixed well using a vortex mixer, and then allowed to stabilize. The fluorescence intensity of the solution was tested. By plotting the fluorescence intensity ratio against the concentration, the critical micelle concentration is determined as the intersection of two tangent lines. The critical micelle concentration of all tested nanoprobes is less than 10 ng/m L. FIG. 2 shows the CMC test results of two typical polymers, the left part shows the test results of PPE90-TPrB, and the right part shows the test results of PPE200-TPrB.

### EXAMPLE 4

### 4.1 Preparation and Characterization of Nanoparticle Solution

5 mg of polymer was dissolved in 0.2 ml CH₃CN, added to 5 ml of deionized water under ultrasonic conditions, concentrated to remove CH₃CN on a rotary evaporator, and supplemented with deionized water until the volume was 5 ml, and the concentration of the obtained stock solution was 1 mg/ml.

### 4.2 DLS Test

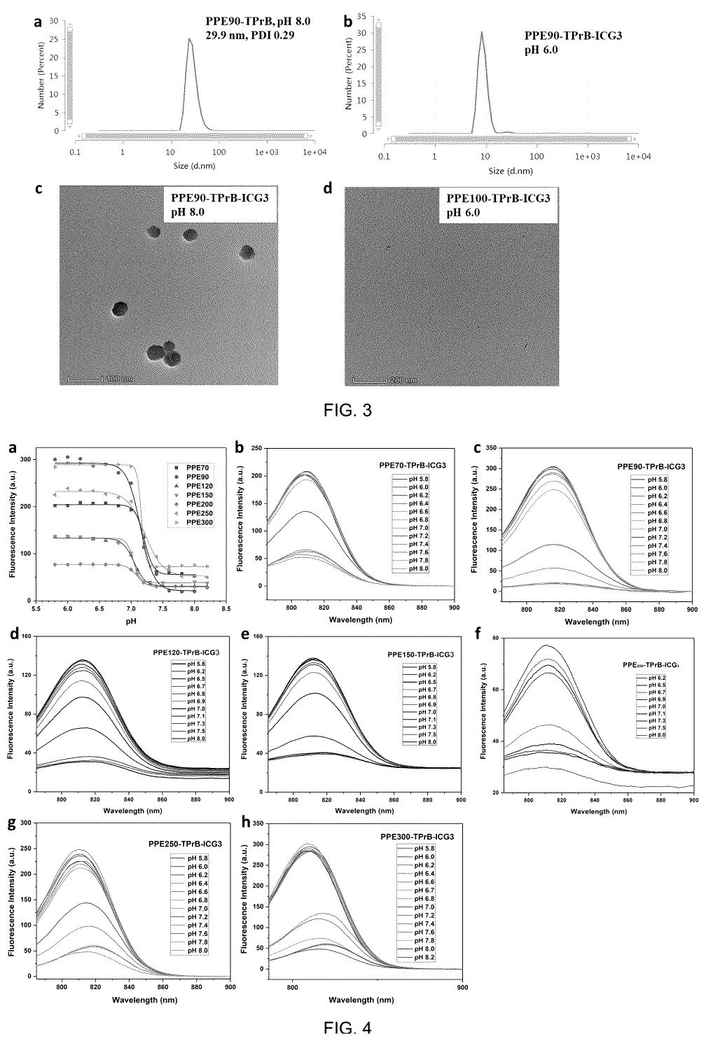

The sample used in this example is the same as that in example 2. PPE90-TPrB was used to prepare the nanoparticle solution. The pH of the solution was about 8.0, the concentration was 1 mg/mL, and the sample was taken at room temperature (20° C.) for DLS (The instrument is: Brookhaven Omni Dynamic Light Scattering (DLS) Particle Sizer and zeta petential Analyzer, all other DLS tests are measured on Malvern Zetasizer Ultra, He-Ne laser, λ=633 nm). The data obtained is shown in FIG. 3a. The nanoparticles are 29.9 nm, and the particle sizes are uniformly distributed.

The nanoparticle solution of example 4.1 was dropped into PBS 6.0, the sample was shaken for 2 minutes and then the DLS test was performed. The data obtained is shown in FIG. 3b, and the polymer nanoparticles are all dissolved.

### 4.3 TEM Test

PPE90-TPrB-ICG was used to prepare the nanoparticle solution. The concentration of this solution is 1 mg/mL, the pH is about 8.0, and the sample is taken for TEM test (ThermoFisher Scientific (formerly FEI), model: Tabs F200S, origin: Netherlands). The data is shown in FIG. 3c. The nanoparticles are about 20-50 nm, and the particle sizes are uniform and are evenly distributed.

The above-mentioned nanoparticle solution was dropped into the PBS6.0 solution for TEM test, and the obtained data is shown in FIG. 3d. Compared with FIG. 3c, the polymer nanoparticles are all dissolved

### EXAMPLE 5

### 5.1 Fluorescence Test

100 uL nanoparticle stock solution (1 mg/mL, refer to example 4.1 for preparation method) was diluted into 2.0 mL of PBS buffer (pH 5.5-8.0), mixed well and emission fluorescence was measured. The excitation light wavelength is 730 nm, and the emission light wavelength detection range is 785-900 nm. The properties of PPE series fluorescent probes are shown in Table 1, where:

pKa and CMC measurement methods refer to Examples 2 and 3.

The calculation of the fluorescence intensity ratio (FIR) is the ratio of the fluorescence intensity at 821 nm in the pH 6.0 buffer solution of the nano fluorescent probe to the fluorescence intensity at 821 nm in the pH 8.0 buffer solution. The calculation method is as follows:

FIR=1821(pH 6.0)/1821(pH 8.0)

Calculation of pH transition point (pHt): Take the fluorescence intensity at 821 nm of different pH values, perform mathematical normalization, and plot the pH versus fluorescence intensity. Then the obtained scatter plot is fitted with boltzmann function. The pH value at 50% fluorescence intensity of the highest fluorescence value is pHt.

pH50%. The calculation method of pH mutation range is as follows:

ΔpH_{10%˜90%}=pH_{10%}−pH_{90%}

TABLE 1
Screening of PPE nano fluorescent probes

| | | Tertiary | | | | | | | |
|---|---|---|---|---|---|---|---|---|---|
| | | amine | Hydrophobic | | | | | | |
| Example | | side | side | ICG/ | | CMC | | | |
| number | DP | chain | chains/number | Chain | pKa | (ng/mL) | FIR | pHt | ΔpH_{10-90%} |
| 1.5.1 | 70 | TPrB | None | 3 | 6.45 | 9.71 | 5.5 | 7.21 | 0.46 |
| 1.5.2 | 90 | TPrB | None | 3 | 6.55 | 8.64 | 19.0 | 7.14 | 0.51 |
| 1.5.3 | 120 | TPrB | None | 3 | 6.53 | 8.85 | 4.3 | 7.05 | 0.44 |
| 1.5.4 | 150 | TPrB | None | 3 | 6.27 | 4.40 | 7.3 | 7.03 | 0.40 |
| 1.5.5 | 200 | TPrB | None | 3 | 6.48 | 4.37 | 10.8 | 7.09 | 0.38 |
| 1.5.8 | 200 | TPrB | C5/40 | 3 | 6.34 | 7.17 | 4.9 | 6.91 | 0.26 |
| 1.5.9 | 200 | TPrB | C9/40 | 3 | 6.24 | 3.56 | 15.2 | 6.75 | 0.22 |
| 1.5.10 | 200 | TPrB | C9/80 | 3 | 6.00 | 1.27 | 9.0 | 6.35 | 0.51 |
| 1.5.6 | 250 | TPrB | None | 3 | 6.42 | 4.42 | 3.8 | 7.22 | 0.49 |
| 1.5.7 | 300 | TPrB | None | 3 | 6.35 | 4.11 | 3.5 | 7.16 | 0.23 |
| 1.5.13 | 90 | TBB | None | 3 | 6.47 | 4.23 | 4.5 | 6.84 | 0.52 |
| 1.5.14 | 90 | TPePe | None | 3 | 5.36 | 3.48 | 7.4 | 5.67 | 0.36 |
| 1.5.15 | 90 | THH | None | 3 | 5.19 | 2.09 | N/A | N/A | N/A |

### 5.2 The Effect of the Degree of Polymerization of the Hydrophobic Block (PPE) on FIR, pHt and ΔpH

The fluorescence test of the nanoparticle stock solution (1 mg/mL, the preparation method refers to example 4.1) was implemented with reference to example 5.1. The relationship between the fluorescence emission intensity (at 821 nm) of PPE-TPrB-ICG3 with different degree of polymerization (DP) and pH is summarized in Table 1 and FIG. 4a. The fluorescence emission spectra of PPE-TPrB-ICG3 with different DP in different PBS buffers are shown in FIGS. 4b-4h.

### 5.3 The Effect of Hydrophobic Side Chains on FIR, pHt and ΔpH of PPE200-TPrB

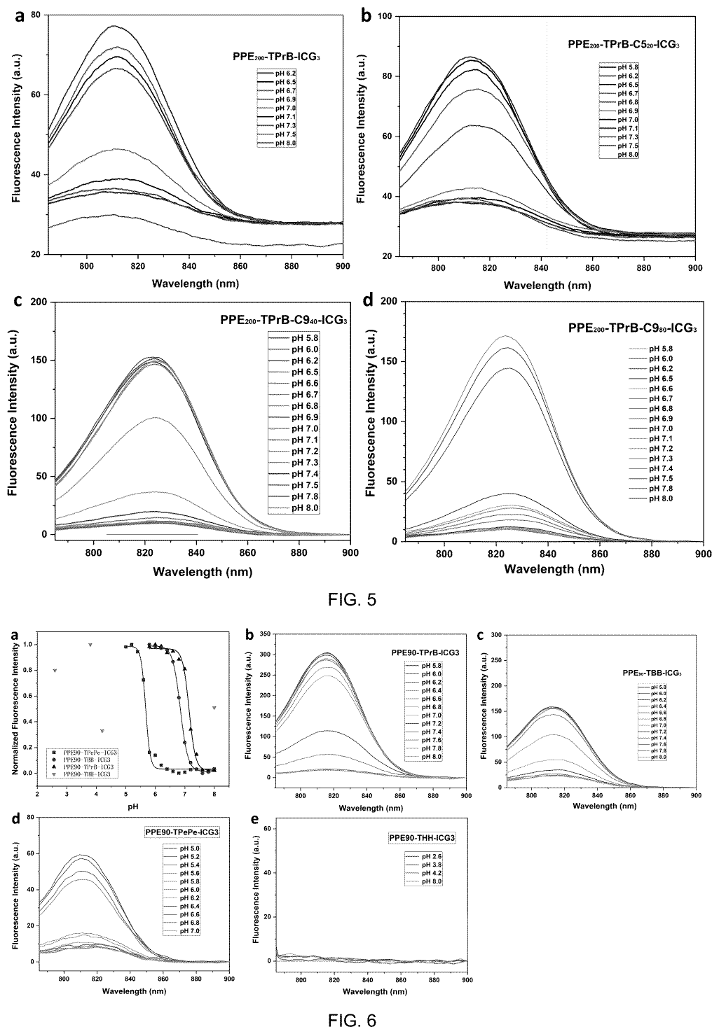

The fluorescence test of the nanoparticle stock solution (1 mg/mL, the preparation method refers to example 4.1) was implemented with reference to example 5.1. The relationship between fluorescence emission spectra and pH of PPE200-TPrB-ICG3 fluorescent probe with 20% C₅H₁₁, 20% C₉H₁₉ or 40% C₉H₁₉ hydrophobic side chain is summarized in Table 1 and FIGS. 5a-5d. When the PPE200 probe is connected with 20% C₅H₁₁ side chain, its pHt and FIR are slightly reduced. When the PPE200 probe is connected with 20% C₉H₁₉ or 40% C₉H₁₉ side chain, its pHt is significantly reduced, and the 20% C₉H₁₉ side chain can improve the FIR. 20% C₅H₁₁ and 20% C9H19 significantly reduced the ΔpH.

### 5.4 The Effect of Side Chain Tertiary Amine on FIR, pHt and ΔpH of PPE200-ICG3

The fluorescence test of the nanoparticle stock solution (1 mg/mL, the preparation method refers to example 4.1) was implemented with reference to example 5.1. The relationship between fluorescence emission spectrum and pH of PPE200-TPrB-ICG3 fluorescent probe with TPrPr, TPrB, TBB, TBPe, TPePe or THH side chain is summarized in Table 1 and FIGS. 6a-6e. With the increase of hydrophobicity of side chain tertiary amine, the pHt of the probe decreased, and the FIR and ΔpH did not change regularly.

As mentioned above, the present disclosure effectively overcomes various shortcomings in the traditional technology and has high industrial utilization value.

The above-mentioned embodiments are merely illustrative of the principle and effects of the present disclosure instead of limiting the present disclosure. Modifications or variations of the above-described embodiments may be made by those skilled in the art without departing from the spirit and scope of the present disclosure. Therefore, all equivalent modifications or changes made by those who have common knowledge in the art without departing from the spirit and technical concept disclosed by the present disclosure shall be still covered by the claims of the present disclosure.

1. A functionalized diblock copolymer, wherein the chemical structure of the functionalized diblock copolymer is shown in Formula I: 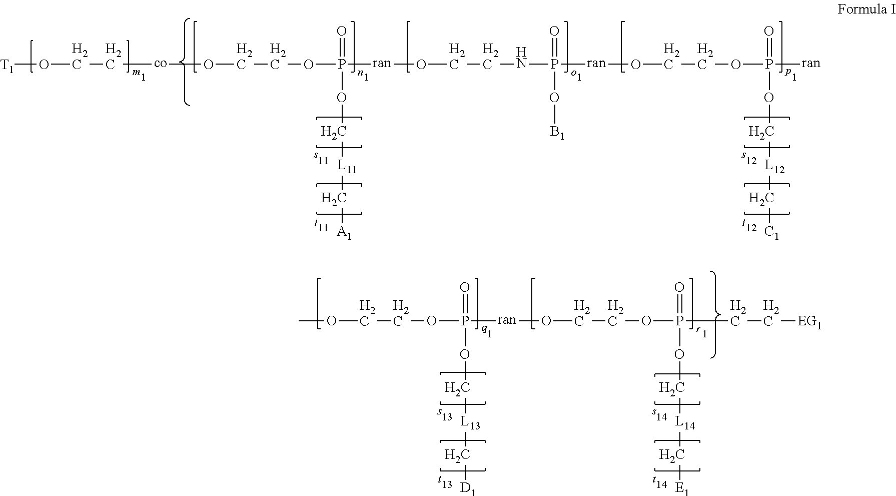
wherein in Formula I, m₁=22˜1136, n₁=10˜500, o₁=0˜50, p₁=0.5˜50, q₁=0˜500 and r₁=0˜200;
s₁₁=1˜10, s₁₂=1˜10, s₁₃=1˜10, s₁₄=1˜10;
t₁₁=1˜10, t₁₂=1˜10, t₁₃=1˜10, t₁₄=1˜10;
L₁₁, L₁₂, L₁₃ and L₁₄ are linking groups;
A₁ is selected from protonatable groups;
B₁ is selected from degradability-regulating groups;
C₁ is selected from fluorescent molecular groups;
D₁ is selected from delivery molecular groups;
E₁ is selected from hydrophilic or hydrophobic groups;
T₁ is selected from capping groups; and
EG₁ is selected from capping groups.

2. The functionalized diblock copolymer of claim 1, wherein in Formula I, a molecular weight of the polyethylene glycol block is in a range of 1,000 to 50,000 Da, and a molecular weight of the polyphosphate block is in a range of 1,000 to 50,000 Da; and/or
a critical micelle concentration (CMC) of the functionalized diblock copolymer is less than 50 μg/mL.

3. The functionalized diblock copolymer of claim 1, wherein in Formula I, s₁₁=1˜5, s₁₂=1˜5, s₁₃=1˜5, s₁₄=1˜5;
t₁₁=1˜6 t₁₂=1˜6, t₁₃=1˜6, t₁₄=1˜6;
L₁₁, L₁₂, L₁₃, L₁₄ are independently selected from —S—, —O—, —OC(O)—, —C(O)O—, —SC(O)—, —C(O)—, —OC(S)—, —C(S)O—, —SS—, —C(R₁)═N—, —N═C(R₂)—, —C(R₃)═N—O—, —O—N═C(R₄)—, —N(R₅)C(O)—, —C(O)N(R₆)—, —N(R₇)C(S)—, —C(S)N(R₈)—, —N(R₉)C(O)N(R₁₀)—, —OS(O)O—, —OP(O)O—, —OP(O)N—, —NP(O)O—, —NP(O)N—, wherein, R₁˜R₁₀ are each independently selected from H, C1-C10 alkyl, and C3-C10 cycloalkyl;
wherein A₁ is 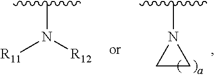
wherein R₁₁ and R₁₂ are each independently selected from C1-C10 alkyl, C2-C10 alkenyl, C2-C10 alkynyl, C3-C10 cycloalkyl, aryl and heteroaryl; a=1-10, and a is a positive integer;
B₁ is selected from C1-C18 alkyl and cation, preferably, the cation is selected from Na⁺, K⁺, Ca²⁺, Zn²⁺, Fe³⁺, Fe²⁺, Li⁺ and NH₄⁺;
C₁ includes one or more of ICG, METHYLENE BLUE, CY3.5, CY5, CY5.5, CY7, CY7.5, BDY630, BDY650, BDY-TMR, Tracy 645 and Tracy 652;
D₁ is selected from fluorescence quenching group and drug molecule group, wherein the fluorescence quenching group is preferably selected from BHQ-0, BHQ-1, BHQ-2, BHQ-3, BHQ-10, QXL-670, QXL-610, QXL-570, QXL 520, QXL-490, QSY35, QSY7, QSY21, QXL 680, Iowa Black RQ and Iowa Black FQ; wherein the drug molecule is preferably selected from chemotherapeutic drugs, more preferably selected from 5-ALA (5-Aminolevulinic acid), nucleic acid drugs, paclitaxel, cisplatin, doxorubicin, irinotecan and SN38;
E₁ is selected from H, C1-C18 alkyl, —O—R₁₁, —S—R₁₂, wherein R₁₁˜R₁₂ are each independently selected from H, C1-C18 alkyl, C3-C10 cycloalkyl, aryl, and heteroaryl;
T₁ is selected from —CH₃ and —H;
EG₁ is selected from —Y—R₁₃, wherein Y is selected from O, S, and N, and R₁₃ is selected from H, C1-C20 alkyl, C3-C10 cycloalkyl, aryl and heteroaryl.

4. The functionalized diblock copolymer of claim 1, wherein in Formula I, m₁=22˜1136, n₁=10˜500, o₁=0, p₁=0.5˜50, q₁=0, r₁=0;
or, in Formula I, m₁=22˜1136, n₁=10˜500, o₁=0, p₁=0.5˜50, q₁=0, r₁=1˜200;
or, in Formula I, m₁=22˜1136, n₁=10˜500, o₁=1˜50, p₁=0.5˜50, q₁=0, r₁=0;
or, in Formula I, m₁=22˜1136, n₁=10˜500, o₁=1˜50, p₁=0.5˜50, q₁=0, r₁=1˜200;
or, in Formula I, m₁=22˜1136, n₁=10˜500, o₁=1˜50, p₁=0.5˜50, q₁=1˜500, r₁=0.

5. The functionalized diblock copolymer of claim 4, wherein the chemical structural formula of the functionalized diblock copolymer is shown as one of the following: 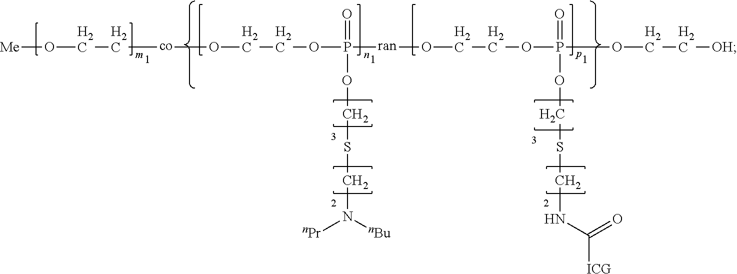
wherein m₁=44˜226, n₁=50˜300, p₁=0.5˜5; 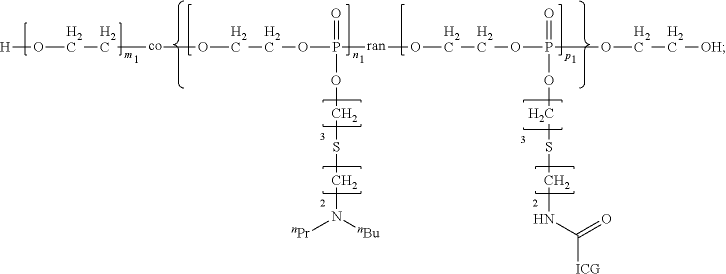
wherein m₁=44˜226, n₁=50˜300, p₁=0.5˜5; 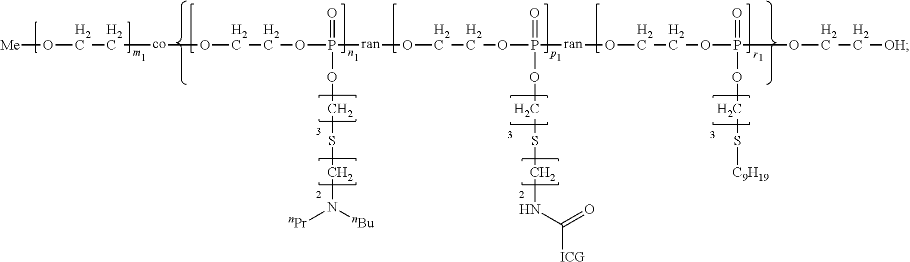
wherein m₁=44˜226, n₁=70˜300, p₁=0.5˜5, r₁=10˜100; 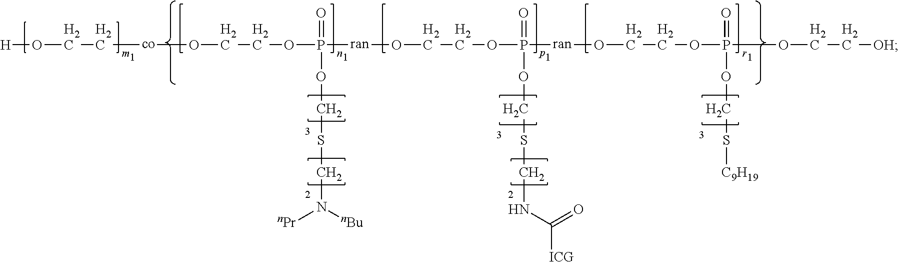
wherein m₁=44˜226, n₁=70˜300, p₁=0.5˜5, r₁=10˜100; 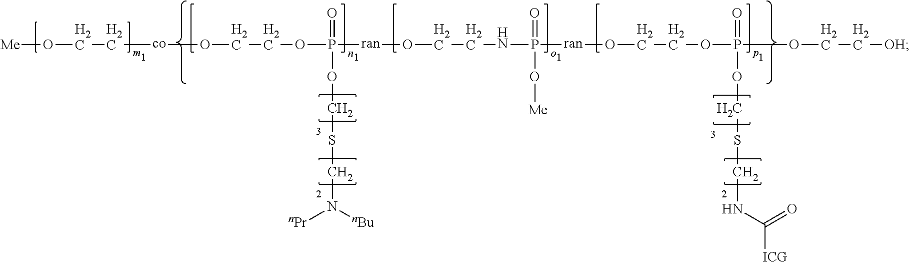
wherein m₁=44˜226, n₁=70˜300, o₁=1˜10, p₁=0.5˜5; 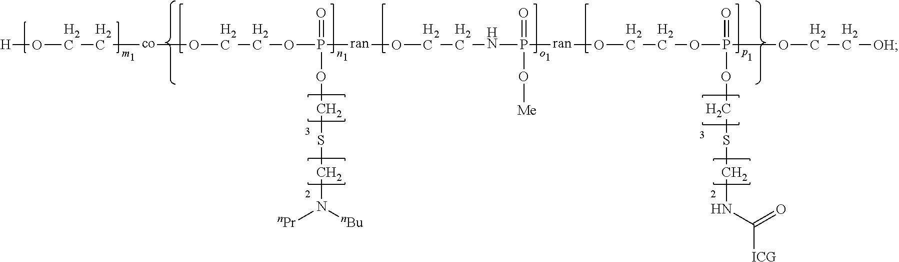
wherein m₁=44˜226, n₁=70˜300, o₁=1˜10, p₁=0.5˜5; 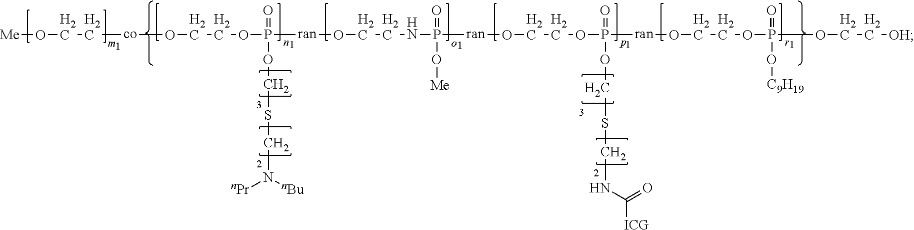
wherein m₁=44˜226, n₁=50˜300, o₁=1˜10, p₁=0.5˜5, r₁=10˜100; 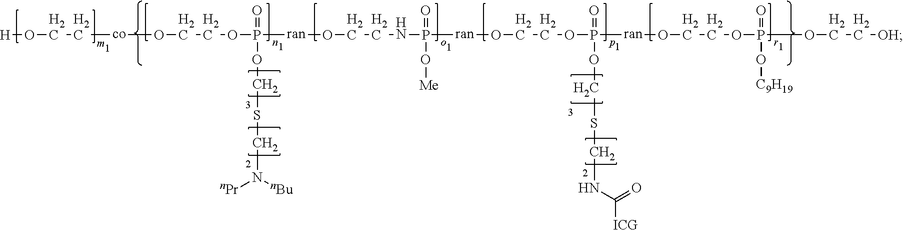
wherein m₁=44˜226, n₁=50˜300, o₁=1˜10, p₁=0.5˜5, r₁=10˜100; 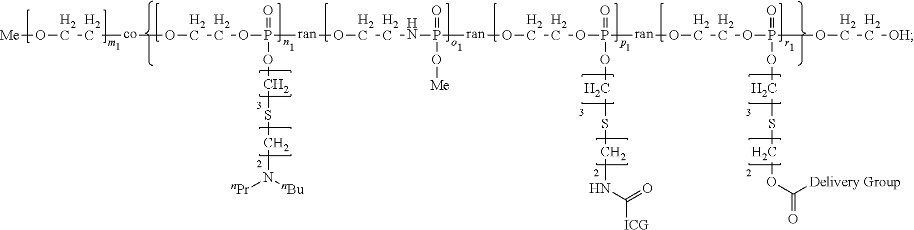
wherein m₁=44˜226, n₁=50˜300, o₁=1˜10, p₁=0.5˜5, q₁=10˜300; 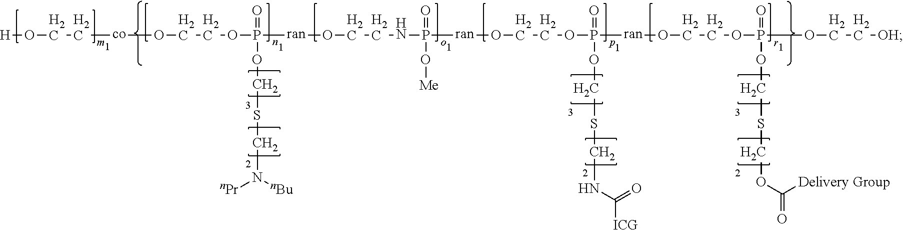
wherein m₁=44˜226, n₁=50˜300, o₁=1˜10, p₁=0.5˜5, q₁=10˜300.

6. Polymer particles, prepared from the functionalized diblock copolymer according to claim 1.

7. The polymer particles of claim 6, wherein a particle size of the polymer particles is in a range of 10 to 200 nm; and/or
the polymer particles are further modified with a targeting group, wherein the targeting group is selected from a group consisting of monoclonal antibody fragments, small molecule targeting groups, polypeptide molecules, and nucleic acid aptamers; and/or,
the targeting group is modified on at least part of a T-terminal of the functionalized diblock copolymer.

8. The functionalized diblock copolymer according to claim 1, or polymer particles prepared from the functionalized diblock copolymer, wherein the functionalized diblock copolymer and/or the polymer particles are degradable in vivo.

9. Use of the functionalized diblock copolymer according to claim 1, or polymer particles prepared from the functionalized diblock copolymer in the preparation of an imaging probe reagent and/or a pharmaceutical preparation, wherein the imaging probe reagent and/or the pharmaceutical preparation preferably has a targeting function, wherein the imaging probe reagent and/or the pharmaceutical preparation is more preferably a targeting imaging probe.

10. A composition, comprising the functionalized diblock copolymer according to claim 1, or polymer particles prepared from the functionalized diblock copolymer.

11. A method of treating or diagnosing a tumor, comprising: administering to an individual an effective amount of the functionalized diblock copolymer according to claim 1, or administering to an individual an effective amount of the polymeric particles prepared from the functionalized diblock copolymer.

12. The method according to claim 11, wherein the functionalized diblock copolymer or the polymeric particles are administered to the individual by administration methods including bladder instillation, uterine perfusion, intestinal perfusion, local administration to the brain after craniotomy, tissue injection during breast cancer dissection surgery, topical administration during minimally invasive surgery for abdominal tumor.
